(19) United States
(12) Patent Application Publication
(10) Pub. No.: US 2010/0015184 A1
(43) Pub. Date: Jan. 21, 2010

(54) Methods of Making Pharmaceutical Components for Customized Drug Products

(76) Inventor: Stephen M. Tuel, Columbia, SC (US)

Correspondence Address: STIPKALA LLC, 5401 NETHERBY LANE, SUITE 102-A, NORTH CHARLESTON, SC, 29420, US

(21) Appl. No.: 12/518,311

(22) PCT Filed: Dec. 13, 2007
(86) PCT No.: PCT/US2007/025430

§ 371 Date: Jun. 9, 2009

Related U.S. Application Data
(60) Provisional application No. 60/874,554, filed on Dec. 13, 2006.

(51) Int. Cl. A61K 9/00

(52) U.S. Cl. 424/400

(57) ABSTRACT

Methods and systems for developing and manufacturing componentized drug product precursor modules that can be simply assembled to create customized drug products are disclosed. Each module contains components of a final drug product (e.g., active pharmaceutical ingredients, nutritional ingredients, and/or excipients) in a fixed mixture selected to maximize desired pharmaceutical characteristics (e.g., stability, manufacturing efficiency) and minimize cost. The modules can be extensively tested for quality and assembled immediately, or at a later time, in multiple combinations to customize the final drug product characteristics (e.g., multiple active ingredients, doses, flavor, viscosity, etc.) to meet individual patient/consumer needs and/or preferences while assuring high quality. Permitted combinations may be maintained in a database to enable networked drug product selection, prescribing, and ordering. Each resulting customized drug product dose can be labeled to facilitate compliance and reduce the number of drug products administered per day.

### CROSS REFERENCE TO RELATED APPLICATIONS

This application claims benefit of priority under PCT Chapter I, Article 8, and 35 U.S.C. § 119(e) of U.S. Provisional Application No. 60/874,554, entitled “Method of Making Pharmaceutical Components for Assembly of Customized Drug Products,” filed on Dec. 13, 2006, which is incorporated herein by reference.

### TECHNICAL FIELD

This invention relates to methods and systems for developing and manufacturing componentized drug product precursor modules that can be assembled to create customized drug products.

### BACKGROUND ART

Pharmaceutical products for use in humans or animals are produced primarily in two ways. High volume or high priced drugs are manufactured under very tightly controlled conditions with detailed specifications, quality testing, and documentation. Because of the high cost and complexity of this type of pharmaceutical manufacturing, low volume, highly specialized drugs are produced in a cottage industry of compounding pharmacies. These pharmacies mix various active ingredients and excipients into an often unique product based on a doctor's prescription. The specifications, quality testing, and documentation are often minimal or nonexistent. Fully testing these products for quality can be cost-prohibitive because of the small volume. Patients are often forced to choose between the assurance of high quality and the flexibility of customized products. When patients are unable to obtain high quality customized drug products they often demonstrate lower compliance with the prescribed regimen and can suffer health consequences.

Processes of preparation and manufacturing of specific active ingredients, including the use of specific excipients in the formulation, are known. These cover only a fraction, however, of the millions of possible combinations of active and inactive ingredients which can address special needs of patient groups. Various compounding machines related to this area are also known, but these merely automate the traditional process of mixing bulk actives with a series of excipients according to a recipe. They do little to address the quality issues inherent in the compounding of medications.

Accordingly, there exists a need for a method and system for developing and manufacturing componentized drug product precursor modules that can be simply assembled to create customized drug products.

### DISCLOSURE OF THE INVENTION

The following presents a simplified summary of the invention in order to provide a basic understanding of some aspects of the invention. This summary is not an extensive overview of the invention. It is not intended to identify key or critical elements of the invention or to delineate the scope of the invention. Its sole purpose is to present concepts of the invention in a simplified form as a prelude to the more detailed description that is presented later.

In some embodiments, the present invention includes a method and system for developing and manufacturing componentized drug product precursor modules that can be simply assembled to create customized drug products. Some precursor modules of the present system contain two or more components of a final drug product in a fixed mixture selected to maximize desired pharmaceutical characteristics and minimize cost. The modules are divided into two classes: 1) modules containing an active pharmaceutical ingredient or specific nutritional ingredient (hereafter called API), and 2) modules containing only excipients.

The modules can be extensively tested for quality and assembled in multiple predetermined combinations, customizing the final drug product characteristics to meet patient/consumer needs and/or preferences while assuring high quality. Permitted combinations can be maintained in an Internet-linkable computer database to enable networked drug product selection, prescribing, and ordering. The resulting customized drug product can facilitate compliance and reduce the number of drug products administered per day.

Some embodiments of the present invention provide an API module, comprising: at least one excipient in a first fixed amount, and at least one active pharmaceutical ingredient in a second fixed amount of at least a dose escalation interval or a fraction thereof. In other embodiments, the second fixed amount can be greater than the dose escalation interval. In still other embodiments, the second fixed amount can be less than a dose of the at least one active pharmaceutical ingredient. Additional embodiments provide the second fixed amount being less than the minimum dose of the at least one active pharmaceutical ingredient. In yet other embodiments, methods for making at least one API module are provided. Such methods comprise, in some embodiments, isolating a first fixed amount of a first API, combining the first fixed amount of the first API with a second fixed amount of at least one first excipient, to form the at least one API module.

Further embodiments provide an excipient module, comprising: at least one first excipient in a first fixed amount; and at least one second excipient in a second fixed amount; wherein the at least one first excipient and the at least one second excipient are compatible with at least one active pharmaceutical ingredient. In some embodiments, the sum of the first fixed amount and the second fixed amount is sufficient to form at least one final drug product comprising the at least one active pharmaceutical ingredient. In other embodiments, the at least one first excipient and the at least one second excipient are compatible with two or more active pharmaceutical ingredients.

Still further embodiments provide a final drug product, made by a process comprising: combining at least one API module comprising at least one active pharmaceutical ingredient, and at least one excipient module, to make the final drug product; wherein the at least one active pharmaceutical ingredient is present in the final drug product in an amount effective for treating at least one disease, at least one disorder, at least one condition, or a combination thereof, in at least one human or animal patient in need thereof.

Yet other embodiments provide a method for manufacturing a final drug product, comprising: selecting a first API module comprising at least one first active pharmaceutical ingredient and at least one first excipient; determining a number of the first API module; choosing a first excipient module comprising at least one second excipient; determining a number of the first excipient module; and combining the number of the first API module and the number of the first excipient module to manufacture the final drug product.

Some embodiments of the present invention further comprise: selecting a second API module comprising at least one second active pharmaceutical ingredient and at least one third excipient; determining a number of the second API module; optionally choosing a second excipient module comprising at least one fourth excipient, and determining a number of the second excipient module; and combining the number of the second API module and optionally the number of the second excipient module with the number of the first API module and the number of the first excipient module to manufacture the final drug product.

Some embodiments of the present invention provide methods of manufacturing at least one final drug product, at least one final drug package, or both, implemented by computer-executable instructions stored on one or more computer-readable media.

Still other embodiments provide a final drug package, comprising: at least one API module, which comprises at least one first excipient, and at least one active pharmaceutical ingredient; and at least one excipient module, which comprises at least one second excipient; wherein the at least one second excipient is compatible with the at least one active pharmaceutical ingredient; wherein the at least one active pharmaceutical ingredient is present in the final drug package in an amount effective for treating at least one disease, at least one disorder, at least one condition, or a combination thereof, in at least one human or animal patient in need thereof; and wherein the at least one API module and the at least one excipient module are adapted to form a final drug product.

Some embodiments provide a system for manufacturing at least one final drug product, at least one final drug package, or both, comprising: at least one API module portfolio comprising a plurality of different API modules; and at least one excipient module portfolio comprising a plurality of different excipient modules; wherein the at least one API module portfolio and the at least one excipient module portfolio are operatively connected to manufacture the at least one final drug product, the at least one final drug package, or both. Other embodiments further comprise one or more means for storing compatibility information for each API module and each excipient module.

Additional embodiments provide a computer-readable medium having computer executable components implementable in a system operable to manufacture at least one final drug product, at least one final drug package, or both, comprising: a computer-executable component for providing one or more of compatibility information, dose information, and dose escalation interval information for at least one API module in an API module portfolio; and a computer-executable component for providing compatibility information for at least one excipient module in an excipient module portfolio. In some embodiments, compatibility information for an API module indicates compatibility with one or more of other APIs, other API modules, excipients, and excipient modules. In still other embodiments, compatibility information for an excipient module indicates compatibility with one or more of APIs, API modules, other excipients, and other excipient modules.

Still other embodiments provide an API module portfolio, comprising a plurality of different API modules. Such embodiments optionally include a computer-readable medium having one or more computer executable components implementable in a system operable to manufacture at least one final drug product, at least one final drug package, or both, comprising: a computer-executable component for providing one or more of compatibility information, dose information, and dose escalation interval information for at least one API module in the API module portfolio. Other embodiments provide such computer-readable medium as a separate embodiment.

Further embodiments provide an excipient module portfolio, comprising a plurality of different excipient modules. Such embodiments optionally include one or more computer-executable components for providing compatibility information for one or more excipient module in the excipient module portfolio. Other embodiments provide such computer-readable medium as a separate embodiment.

Other embodiments provide methods for developing an API module portfolio. Still other embodiments provide methods for developing an excipient module portfolio.

Other features and advantages of the present invention will be apparent to those skilled in the art from a careful reading of the entire application, including the Modes for Carrying Out the Invention presented below and accompanied by the drawings.

### BRIEF DESCRIPTION OF DRAWINGS

FIG. 1 shows a process for selection of an API module dose according to one embodiment of the present invention;

FIG. 2 shows a process for minimizing the number of excipients in an API module according to one embodiment of the present invention;

FIG. 3 shows a process for developing a portfolio of, and selecting the components in, modules not containing an API according to one embodiment of the present invention;

FIG. 4 shows a process for managing the portfolio of modules according to one embodiment of the present invention;

FIG. 5 shows a process for selecting modules for final drug assembly according to one embodiment of the present invention;

FIG. 6 shows a process including alternative steps for assembling modules for the final drug product according to one embodiment of the present invention;

FIG. 7 shows a method for controlling quality, including assessing compatibility and permissible combinations of modules, and ordering processes according to one embodiment of the present invention.

### MODES FOR CARRYING OUT THE INVENTION

“API module” indicates an isolated first fixed amount of at least one active pharmaceutical ingredient together with a second fixed amount of at least one excipient. In certain embodiments, the at least one excipient is compatible with the at least one API. The API module may be enclosed in any suitable container, for example, so that the module may be stored for a period of time before being combined with one or more other modules to form a final drug product. Suitable containers include, but are not limited to, vials, ampules, capsules, pipettes, packets such as foil packets, bubble packets, and the like; and those containers may be water soluble, soluble in another solvent, insoluble, biodegradable, edible, recyclable, reusable, sterilizable, disposable, or combinations thereof. Such containers can be made of any suitable material, such as, for example, glass, metal, ceramic, polymer, paper, cardboard, and combinations thereof. In some embodiments, an API module is not enclosed in a container; in such embodiments, the API module could be in the form of a pill, pellet, frozen pellet, single granule, or the like. The first fixed amount of the at least one API in an API module is any suitable amount, and is known or is determinable. In some embodiments, the first fixed amount is equal to the dose escalation interval for the API, the minimum dose for the API, or both when those amounts are equal. In other embodiments, the first fixed amount is a fraction of the dose escalation interval or the minimum dose. In still other embodiments, the first fixed amount is an amount other than the dose escalation interval, the minimum dose, or a fraction thereof. For example, the first fixed amount could be a multiple of the minimum dose or the dose escalation interval, or it could be any other suitable amount. In some embodiments, the first fixed amount does not exceed the maximum dose of the API. In other embodiments, the first fixed amount does not exceed the maximum safe amount of the API. In still other embodiments, the first fixed amount does not exceed about 80%, about 50%, about 25%, or about 10% of the minimum dose of the API. The second fixed amount of the at least one excipient is any suitable amount. In some embodiments, the second fixed amount is an amount sufficient to carry, solubilize, suspend, bind, and/or stabilize the first fixed amount of the at least one API, and/or impart at least one characteristic to a final drug product. In still other embodiments, an API module optionally contains at least one second API in a third fixed amount. In further embodiments, an API module contains at least one second excipient in a fourth fixed amount. The concept of the fixed amount allows for degradation of a given ingredient that may occur over time in some embodiments.

“Excipient module” indicates an isolated first fixed amount of at least one excipient. The first fixed amount can be any suitable amount. In some embodiments, the first fixed amount is known or is determinable. In other embodiments, the first fixed amount is a sufficient amount, that is, an amount sufficient to carry, solubilize, suspend, bind, and/or stabilize a given amount of at least one API and/or at least one other excipient, and/or impart at least one characteristic to a final drug product. The excipient module can comprise a container, in some embodiments, such as those containers described for API modules. In other embodiments, an excipient module comprises a first fixed amount of a first excipient, and a second fixed amount of a second excipient. The first fixed amount and the second fixed amount are, independently, any suitable amounts. In some embodiments, the first fixed amount and the second fixed amount together are sufficient to carry, solubilize, suspend, bind, and/or stabilize a given amount of an API, and/or impart at least one characteristic to a final drug product. In other embodiments, the first fixed amount and the second fixed amount together are sufficient to carry, solubilize, suspend, bind, and/or stabilize various amounts of more than one API, and/or impart at least one characteristic to a final drug product. In still other embodiments, the first fixed amount is sufficient to carry, solubilize, suspend, bind, and/or stabilize a given amount of a first API, while the second fixed amount is sufficient to carry, solubilize, suspend, bind, and/or stabilize another given amount of a second API. In still other embodiments, an excipient module comprises a first excipient in a first fixed amount effective to impart a characteristic to a final drug product, and a second excipient in a second fixed amount effective to carry, solubilize, suspend, bind, and/or stabilize the first excipient in the first fixed amount. Excipient modules may contain two or more excipients for any suitable purpose in any suitable amounts. In some embodiments, those suitable amounts do not exceed amounts needed to form the maximum dose of an API, or the maximum safe amount of an API in administrable form. In other embodiments, those suitable amounts are fractions of the amounts sufficient to carry, solubilize, suspend, bind, and/or stabilize a dose of an API, and/or impart at least one characteristic to a final drug product. Those suitable amounts can be chosen from amounts suitable for a minimum dose of an API, a dose escalation interval for an API, or a fraction of either of the foregoing, in some embodiments.

“API module portfolio” refers to a plurality of different API modules. In some embodiments, information about one or more API modules is available for the API module portfolio. Such information includes any suitable information, and may comprise one or more of identity, amount, co-API compatibility, excipient compatibility, co-medication compatibility (for APIs administered separately), allergy and adverse drug reaction information, minimum dose, dose escalation interval, dosing schedule, and maximum dose for each API in each API module.

“Excipient module portfolio” indicates a plurality of different excipient modules. In some embodiments, information about one or more excipient modules is available for the excipient module portfolio. Such information includes any suitable information, and may comprise one or more of identity, amount, API compatibility, co-excipient compatibility, allergy information, minimum effective excipient amounts, maximum recommended excipient amounts, dosage form information, timed-release information, and the like for each excipient in each excipient module in the portfolio.

“Final drug package” means a collection of one or more API modules and one or more excipient modules sufficient to formulate a final drug product. In some embodiments, a final drug package is adapted to be provided to a treating medical professional and/or a patient so the professional or patient can formulate the final drug product. In further embodiments, a final drug package is sufficient to formulate a single dose. In still further embodiments, a final drug package is sufficient to independently formulate more than one single dose. For example, a final drug package in some embodiments contains enough modules and optionally instructions to formulate a month's worth of daily single doses. A final drug package, in certain embodiments, is not in a form intended for administration directly to the patient.

“Final drug product” means, in some embodiments, a combination of the contents of at least one API module and at least one excipient module in a form that can be administered to a human or animal patient. In some embodiments, a final drug product contains one dose of at least one API, and a sufficient amount of at least one excipient. In other embodiments, a final drug product comprises the contents of at least one API module and two or more alike or different excipient modules. In still other embodiments, a final drug product comprises the contents of two or more alike or different API modules and two or more alike or different excipient modules.

In those embodiments in which “the excipients are the same or different,” that means, for example, that a first excipient can be the same as, or different from, a second excipient. If there are more than two excipients, any one excipient may be the same as or different from any other excipient. For example, a first excipient may be the same as a second excipient, both of which differ from a third excipient. Alternatively, all three excipients in the previous example can differ from each other. The amount of any excipient in one module is independent of whether the excipient is the same as or different from any other excipient in another module.

“Combining the number” of modules refers to combining the contents of the modules, which are present in any suitable number, in any suitable manner. The modules could be mixed together, and their biodegradable or edible containers are mixed in with the contents of the modules, in some embodiments. In other embodiments, the contents of the modules are removed from the containers and mixed together. In certain embodiments, the contents of the modules are quantitatively transferred for mixing. In still other embodiments, the contents of some modules could be added to the contents of other modules, in the containers of those other modules.

“Manufacture” can indicate any suitable steps for making an article. Manufacturing can take place in a factory, in a specially-adapted apparatus such as a kiosk, a portable kit, and a machine, and in a patient's home. For example, a patient can manufacture a final drug product from a final drug package by combining a number of alike or different API modules and a number of alike or different excipient modules to form the final drug product.

The present invention provides, in some embodiments, a method and system for developing and manufacturing componentized drug product precursor modules. The modules are divided into two classes: 1) modules containing an active pharmaceutical ingredient or specific nutritional ingredient (API), and 2) modules containing only excipients. The development process differs for each class.

### Modules Containing an API

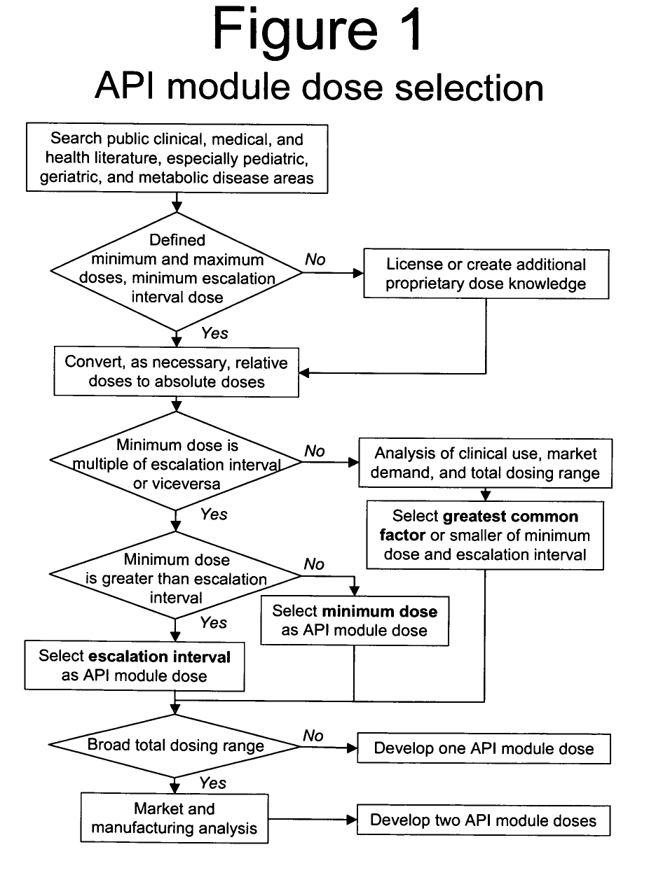

Some issues that govern the development of modules that contain an API include selecting the dose and minimizing the number of excipients. FIG. 1 illustrates and summarizes the process of selecting the dose of an API in the module, in one embodiment of the invention. One possible step in determining the dose of an API module is to assess the range of doses prescribed and/or used for that particular API. Drug formularies and FDA records may provide information on currently manufactured APIs, in some embodiments. In other embodiments, recommended minimum and maximum doses, and minimum dose escalation intervals, can be determined through a detailed assessment of the clinical, medical, and health literature by methods known in the art. Searches need not be limited to specific diseases, conditions, or indications, but may include all potential uses, both regulatory-approved and unapproved. Searches for minimum dose and dose escalation interval may focus on literature discussing pediatric, geriatric, and metabolic disease (e.g., hepatic insufficiency) because these populations are often more fragile and require greater accuracy in dosing. In cases where the public literature is insufficient, it may be necessary to license or otherwise obtain existing proprietary knowledge or to create proprietary knowledge through clinical trial methods known in the art.

Dose recommendations are often given in terms relative to patient characteristics, especially in pediatric patients, e.g., a dose may be relative to the patient's weight or body surface area and recommended as a number of milligrams per kilogram or square meter respectively. In these cases, the dose recommendation must be converted into an absolute measure by evaluating the applicable characteristic of the patient population using the particular API. For example, an API given to adolescents for hormonal regulation during puberty is unlikely to be used in a patient weighing less than 30 kg. If the relative minimum dose is 8 mg/kg and the dose escalation interval is 2 mg/kg, then the absolute minimum dose would be 240 mg and the dose escalation interval would be 60 mg. An API with identical relative dose recommendations (8 mg/kg and 2 mg/kg respectively) that is used for life support in premature infants (who can weight only hundreds of grams) would have much smaller absolute dose recommendations at 4 mg and 1 mg for the minimum and escalation interval respectively. In some embodiments, a “dose” is an amount of an API effective for treating at least one disease, at least one disorder, at least one condition, or a combination thereof. In other embodiments, a “dose” is an established amount of an API, such as, for example, a recommended daily allowance.

Once absolute minimum and maximum dose, and minimum dose escalation interval are established, the dose of the API within the module is selected. In some embodiments, the minimum dose is either equal to, or is a whole number multiple of, the dose escalation interval, e.g., for a dose escalation interval of 5 mg, the minimum dose is usually 5 mg, or 10 mg, or 15 mg, etc. Also, in some cases, the minimum escalation interval is less than the minimum dose and all generally prescribed doses will be a whole number multiple of the dose escalation interval. Thus, in some API modules, the dose in a single module is equal to the minimum dose escalation interval.

In the event that the dose escalation interval is greater than the minimum recommended dose, the minimum recommended dose may be selected for the dose within the API module if the dose escalation interval is a whole number multiple of that minimum dose (e.g., for a minimum dose of 10 mg, and a dose escalation interval of 20 mg, or 30 mg, etc.).

Another potential circumstance is where the minimum dose is not a whole number multiple of the dose escalation interval, e.g., a minimum dose of 5 mg with a dose escalation interval of 2 mg. In this case a judgment must be made whether to use the greatest common factor of the minimum dose and escalation interval (e.g., 1 mg in the previous example), or to proceed with the smaller of the minimum dose and escalation interval (2 mg in the previous example) and force prescribing clinicians and/or consumers to choose between a dose somewhat higher or lower than the recommended minimum (e.g., 4 mg or 6 mg in the previous example), or to choose another amount. This decision may rely on a broad analysis of clinical usage (e.g., physicians may prefer the somewhat lower minimum dose), market demand (e.g., the percentage of patients actually using the minimum dose may be insignificant; in the previous example every patient may be receiving 10 mg or above), and total dosing range (i.e., smaller modular dose is more practical for a narrow total range, as will be discussed below).

Another API dose selection decision is whether to develop an additional dose module, such as, for example, an API module having a higher dose of the API. In drugs with a broad total range (maximum dose minus minimum dose), the maximum dose can be a very large multiple of the minimum dose or escalation interval. This can require a large number of API modules, e.g., a drug with a minimum dose/escalation interval of 5 mg, yielding an API module dose of 5 mg, would require 100 modules to reach a maximum dose of 500 mg. For some embodiments, a large number of API modules may be considered inconvenient. In these cases one or more higher dose modules can be developed. The exact dose multiple of the initial module may depend on market and manufacturing analysis, but a 10× multiple is a useful default option for some embodiments.

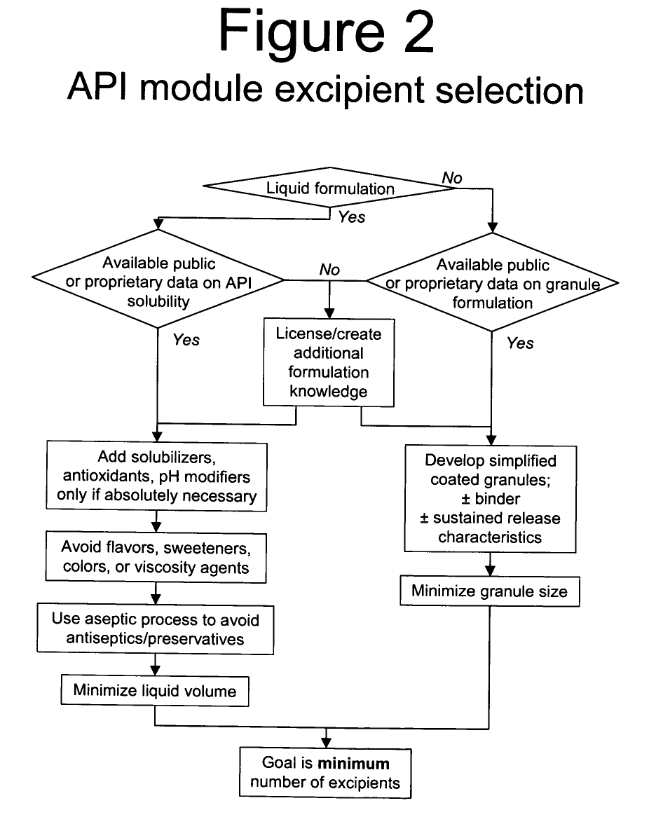

FIG. 2 summarizes the process of minimizing the number of excipients in an API module, in one embodiment. After the dose of an API module has been selected, the next step can be to choose one or more additional components to carry and stabilize the active ingredient within the module. For some embodiments such as solid formulations, this may be a relatively straightforward process of encapsulating very small amounts of the API, with or without a binder, in a water-soluble coating, with or without sustained release characteristics, using methods known in the art to create granules such as those used to fill capsules. The granule size may be minimized to permit the greatest number of potential combinations during final assembly of the final drug product.

For some embodiments comprising liquid formulations, the process may start with the selection of a solvent or suspending agent to create a liquid. A liquid formulation may be in any suitable form, such as solution, suspension, slurry, or the like. Some liquid formulation API modules of the present invention provide formulary simplicity. In certain embodiments, an API module has only an active ingredient and a solvent; whenever possible additional excipients may be avoided. Antioxidants, acidifiers, alkalinizers, and buffers may be required, but for an API module, no flavors, sweeteners or taste-masking agents, colors, or viscosity agents are needed in some embodiments. Preservatives and antiseptics are permitted, but can be avoided by using an aseptic manufacturing process. Minimizing the number of excipients may simplify analytic method development and manufacturing development and expense, and likely promotes a more stable formulation. Based on these and/or other considerations, the formula is developed by methods known in the art. The volume of liquid can be minimized to permit the greatest number of potential combinations during assembly of the final drug product.

APIs may be chosen from any suitable active pharmaceutical ingredient. As used herein, “active pharmaceutical ingredient” refers very broadly to any substance that may have pharmaceutical, nutritional, nutraceutical, or other activity in a human or animal body. Such substances need not require formal government approval, such as FDA approval. Substances that can be sold as dietary supplements, such as vitamins, minerals, botanicals, and the like are specifically included within the scope of APIs. In some embodiments, an active pharmaceutical ingredient is any component of a final drug product intended to furnish pharmacological activity or other direct effect in the diagnosis, cure, mitigation, treatment, or prevention of disease, or to affect the structure or any function of the body of humans or other animals. Active pharmaceutical ingredients include those components of the final drug product that may undergo chemical change during the manufacture of the final drug product and be present in the final drug product in a modified form intended to furnish the specified activity or effect. In some embodiments, active pharmaceutical ingredients include substances that affect the general health and/or aging of one or more body systems. Active pharmaceutical ingredients include prodrugs, metabolites, stereoisomers, enantiomers, diastereomers, salts, solvates, hydrates, complexes, polymorphs, crystals, co-crystals, and other chemical- and physical derivatives of pharmacologically-active substances.

Moreover, it is understood that some substances may be considered active, and may also find use as an excipient. For example, certain antibiotics could be useful as APIs in some embodiments of the present invention, while they also could find use as preservative excipients in other embodiments of the present invention. Indeed, some ingredients could function simultaneously as both APIs and excipients. For further examples, alcohol and mineral oil can be used as APIs or as excipients. One of ordinary skill in the art will appreciate that describing a substance as a member of one class (e.g., API) does not preclude using that substance in the other class (e.g., excipient), and vice versa.

Active pharmaceutical ingredients suitable for API modules include, but are not limited to, analgesics and muscle relaxants, anesthetics and surgical adjuvants, antibiotics, antidiabetic agents, antifungal agents, antihistamines, anti-inflammatory agents, antimicrobial agents, antiparasitic agents, antivirals, biological and cellular agents, bone disease agents, botanicals, cardiovascular agents, dermatologic agents, diuretics, gastrointestinal agents, hematologic and oncologic agents, hormones, minerals, neuromodulators, nutraceuticals, obstetric and fertility agents, opthalmologic agents, proteins, enzymes and enzyme inhibitors, respiratory agents, rheumatologic agents, steroids, urological agents, vitamins, and other agents.

Suitable analgesics and muscle relaxants include, but are not limited to, acetaminophen, alfentanil, almotriptan, aspirin, bromfenac, buprenorphine, butalbital, butorphanol, carisoprodol, chlorzoxazone, codeine, cyclobenzaprine, dantrolene, dihydrocodeine, dihydroergotamine, eletriptan, ergotamine, fentanyl, frovatriptan, hydrocodone, hydromorphone, levorphanol, meclofenamate, meprobamate, meperidine, metaxalone, methadone, methocarbamol, morphine, nabilone, nalbuphine, naratriptan, pentazocine, orphenadrine, oxycodone, oxymorphone, propoxyphene, remifentanil, rizatriptan, sufentanil, sumatriptan, tramadol, ziconotide, and zolmitriptan.

Suitable anesthetics and surgical adjuvants include, but are not limited to, articaine, atracurium, benzocaine, bupivacaine, chloroprocaine, cisatracurium, desflurane, dexmedetomidine, dibucaine, enflurane, etomidate, glycopyrrolate, isoflurane, ketamine, lidocaine, mepivacaine, metaraminol, methohexital, midazolam, pancuronium, prilocalne, procaine, proparacaine, propofol, rocuronium, ropivacaine, sevoflurane, succinylcholine, tetracaine, and vecuronium.

Suitable antibiotics include, but are not limited to, acetohydroxamic acid, amikacin, aminosalicylic acid, amoxicillin, ampicillin, azithromycin, aztreonam, bacitracin, bleomycin, capreomycin, carbenicillin, cefaclor, cefadroxil, cefazolin, cefdinir, cefditoren, cefepime, cefixime, cefoperazone, cefotaxime, cefotetan, cefoxitin, cefpodoxime, cefprozil, ceftazidime, ceftibuten, ceftizoxime, ceftriaxone, cefuroxime, cephalexin, chloramphenicol, ciprofloxacin, clarithromycin, clindamycin, cloxacillin, colistin, cycloserine, dapsone, daptomycin, demeclocycline, dicloxacillin, doripenem, doxycycline, ertapenem, erythromycin, ethambutol, ethionamide, fosfomycin, gemifloxacin, gentamicin, gramicidin, imipenem, isoniazid, kanamycin, levofloxacin, lincomycin, linezolid, lomefloxacin, mafenide, meropenem, methenamine, minocycline, moxifloxacin, mupirocin, nafcillin, nalidixic acid, neomycin, nitrofurantoin, nitrofurazone, norfloxacin, ofloxacin, oxacillin, oxytetracycline, penicillin, piperacillin, polymyxin B, pyrazinamide, retapamulin, rifabutin, rifampin, rifapentin, rifaximin, spectinomycin, streptomycin, sulbactam, sulfacetamide, sulfadiazine, sulfamethoxazole, sulfanilamide, sulfisoxazole, telithromycin, tetracycline, ticarcillin, tigecycline, tobramycin, trimethoprim, and vancomycin.

Suitable antidiabetic agents include, but are not limited to, acarbose, acetohexamide, chlorpropamide, exenatide, glimepiride, glipizide, glyburide, insulin, levocarnitine, mefformin, miglitol, nateglinide, pioglitazone, pramlintide, repaglinide, rosiglitazone, sitagliptin, thiazolidinedione, tolazamide, and tolbutamide.

Suitable antifungal agents include, but are not limited to, amphotericin B, anidulafungin, butenafine, butoconazole, caspofungin, ciclopirox, clioquinol, clotrimazole, econazole, fluconazole, flucytosine, griseofulvin, itraconazole, ketoconazole, metronidazole, micafungin, miconazole, naftifine, natamycin, nystatin, oxiconazole, posaconazole, sertaconazole, sulconazole, terbinafine, terconazole, tioconazole, and voriconazole.

Suitable antihistamines include, but are not limited to, acrivastine, antazoline, azelastine, bromazine, brompheniramine, carbinoxamine, cetirizine, chlorpheniramine, clemastine, cromolyn, cyproheptadine, desloratadine, dexbrompheniramine, dexchlorpheniramine, dimenhydrinate, diphenhydramine, doxylamine, emedastine, epinastine, ethanolamine, fexofenadine, hydroxyzine, ketotifen, levocetirizine, loratadine, meclizine, olopatadine, pheniramine, promethazine, and triprolidine.

Suitable anti-inflammatory agents include, but are not limited to, amlexanox, celecoxib, clofazimine, diclofenac, diflunisal, dimethyl sulfoxide, etodolac, fenoprofen, flurbiprofen, hydrocortisone, ibuprofen, indomethacin, ketoprofen, ketorolac, mefenamic acid, meloxicam, nabumetone, naproxen, nepafenac, oxaprozin, piroxicam, sulindac, tolmetin, and tromethamine.

Suitable antimicrobial agents include, but are not limited to, benzalkonium, benzethonium, chlorhexidine, chloroxylenol, hexachlorophene, malathion, resorcinol, thimerosal, triclocarban, and triclosan.

Suitable antiparasitic agents include, but are not limited to, albendazole, atovaquone, chloroquine, diatrizoate, eflornithine, hydroxychloroquine, iodoquinol, ivermectin, lindane, mebendazole, mefloquine, metronidazole, nitazoxanide, paromomycin, pentamidine, permethrin, piperonyl butoxide, praziquantel, primaquine, proguanil, pyrimethamine, and tinidazole.

Suitable antivirals include, but are not limited to, abacavir, acyclovir, adefovir, amantadine, amprenavir, atazanavir, cidofovir, darunavir, delavirdine, didanosine, docosanol, efavirenz, emtricitabine, enfuvirtide, entecavir, famciclovir, fosamprenavir, foscarnet, ganciclovir, idoxuridine, indinavir, lamivudine, lopinavir, maraviroc, nelfinavir, nevirapine, oseltamivir, penciclovir, raltegravir, ribavirin, rimantadine, ritonavir, saquinavir, stavudine, telbivudine, tenofovir, thiabendazole, tipranavir, trifluridine, valaciclovir, valganciclovir, zalcitabine, zanamivir, and zidovudine.

Suitable biological and cellular agents include, but are not limited to, antibodies, antigens, bacteria, cell fragments, DNA, molds, phages, polynucleotides, polypeptides, RNA, stem cells, viruses, whole cells, and yeasts.

Suitable bone disease agents include, but are not limited to, alendronate, calcitonin, etidronate, ibandronate, pamidronate, raloxifene, risedronate, tiludronate, and zoledronate.

Suitable botanicals include, but are not limited to, agrimony, alfalfa, algae (red, brown, and green), aloe vera, anise, apple, ashwagandha, astragalus, barberry, barley, basil, bilberry, blackberry, black cohosh, black currant, black haw, black walnut, bloodroot, boneset, borage, boswellia, broom, brussel sprouts, bugleweed, burdock, butcher's broom, cabbage, calendula, camphor, cantaloupe, capsicum, caraway, cardamom, cascara sagrada, catnip, cat's claw, cayenne, celery, chamomile, chaste berry, chicory, Chinese cucumber, choke cherry, cinnamon, cleavers, cloves, cocoa, coltsfoot, comfrey, coriander, cramp bark, cranberry, dandelion, devil's claw, dill, dong quai, echinacea, elder, eucalyptus, evening primrose, fennel, fenugreek, feverfew, flax, fig, garlic, gentian, ginger, ginkgo, ginseng, goldenrod, goldenseal, gotu kola, grape seed, grapefruit, guarana, guggul, hawthorn, hops, horehound, horse chestnut, horseradish, horsetail, hyssop, Iceland moss, ipecac, jasmine, jojoba, kava, kelp, kombucha mushroom, lady's mantle, lavender, legumes, lemon, lemon balm, licorice, lime, lovage, mango, marijuana, marshmallow, meadowsweet, milk thistle, mint, motherwort, mullein, mustard, myrrh, nettle, oak, oat bran, olive, onion, orange, oregano, papaya, parsley, passionflower, pear, peppermint, pineapple, plum, pomegranate, poppy, pumpkin seed, raspberry (red and black), red clover, red rice yeast, rose, rosemary, safflower, sage, St. John's wort, savory, saw palmetto, seneca root, senna, sesame, slippery elm, soapwort, star anise, strawberry, tea tree, thyme, tomato, turmeric, uva ursi, valerian, vervain, white willow, wild yam, wintergreen, witch hazel, yarrow, yellow dock, and the extracts of any of the foregoing.

Suitable cardiovascular agents include, but are not limited to, acebutolol, aliskiren, ambrisentan, amiodarone, amlodipine, aprotinin, atenolol, atorvastatin, benazepril, betaxolol, bisoprolol, bosentan, bretylium, candesartan, captopril, carteolol, carvedilol, cilostazol, clofibrate, clonidine, clopidogrel, diazoxide, digoxin, diltiazem, dipyridamole, disopyramide, dobutamine, dofetilide, doxazosin, enalapril, enalaprilat, epinephrine, eplerenone, epoprostenol, eprosartan, eptifibatide, esmolol, ezetimibe, felodipine, fenofibrate, fenoldopam, flecainide, fluvastatin, fosinopril, gemfibrozil, guanabenz, guanfacine, hydralazine, ibutilide, iloprost, inamrinone, irbesartan, isoetharine, isoproterenol, isosorbide, isradipine, labetalol, levobunolol, lisinopril, losartan, lovastatin, mecamylamine, methyclothiazide, methyldopa, metoprolol, metyrosine, mexiletine, midodrine, milrinone, minoxidil, moexipril, nadolol, naphazoline, nesiritide, nicardipine, nifedipine, nimodipine, nisoldipine, nitroglycerin, norepinephrine, olmesartan, penbutolol, pentoxifylline, perindopril, phenoxybenzamine, phentolamine, pindolol, prazosin, pravastatin, procainamide, propafenone, propranolol, quinapril, quinidine, ramipril, ranolazine, rescinnamine, reserpine, rosuvastatin, sildenafil, simvastatin, sotalol, tadalafil, telmisartan, tetrahydrozoline, ticlopidine, tirofiban, trandolapril, trepostinil, valsartan, vardenafil, verapamil, and xylometazoline.

Suitable dermatologic agents include, but are not limited to, acitretin, adapalene, alitretinoin, allantoin, alpha hydroxy acid, anthralin, avobenzone, azelaic acid, bentoquatam, benzoyl peroxide, calcipotriene, clobetasol, crotamiton, desonide, desoximetasone, dioxybenzone, ecamsule, fluocinonide, flurandrenolide, halcinonide, hydroquinone, isotretinoin, mequinol, methoxsalen, methyl aminolevulinate, monobenzone, octinoxate, octocrylene, oxybenzone, pimecrolimus, pramoxine, prednicarbate, sinecatechins, tazarotene, titanium dioxide, tretinoin, and zinc oxide.

Suitable diuretics include, but are not limited to, acetazolamide, amiloride, bendroflumethiazide, bumetanide, chlorothiazide, chlorthalidone, ethacrynate, furosemide, hydrochlorothiazide, hydroflumethiazide, indapamide, methazolamide, metolazone, polythiazide, spironolactone, torsemide, and triamterene.

Suitable gastrointestinal agents include, but are not limited to, alosetron, aluminum hydroxide, aprepitant, balsalazide, bisacodyl, bismuth subsalicylate, cholestyramine, cimetidine, colesevelam, colestipol, dicyclomine, difenoxin, diphenoxylate, dronabinol, esomeprazole, famotidine, lactulose, lansoprazole, loperamide, lubiprostone, mepenzolate, mesalamine, methscopolamine, metoclopramide, misoprostol, nizatadine, olsalazine, omeprazole, ondansetron, orlistat, pantoprazole, rabeprazole, ranitidine, scopolamine, simethicone, sincalide, sucralfate, sulfasalazine, trimethobenzamide, and ursodiol.

Suitable hematologic and oncologic agents include, but are not limited to, altretamine, amifostine, aminoglutethimide, aminolevulinic acid, anagrelide, anastrozole, argatroban, azacytidine, bexarotene, bicalutamide, bivalirudin, bortezomib, busulfan, capecitabine, carboplatin, carmustine, cetrorelix, chlorambucil, cisplatin, cladribine, clofarabine, cyclophosphamide, cyclosporine, cytarabine, dacarbazine, dactinomycin, dalfopristin, dalteparin, dasatinib, daunorubicin, decitabine, deferasirox, deferoxamine, desirudin, dexrazoxane, docetaxel, dolasetron, doxorubicin, enoxaparin, epirubicin, erlotinib, estramustine, etoposide, exemestane, floxuridine, fludarabine, fluorouracil, flutamide, fondaparinux, fulvestrant, gefitinib, gemcitabine, gemtuzumab, granisetron, heparin, hydroxyurea, idarubicin, ifosfamide, imatinib, imiquimod, irinotecan, ixabepilone, lapatinib, lenalidomide, lepirudin, letrozole, leucovorin, mechlorethamine, megestrol, melphalan, mercaptopurine, mesna, methotrexate, mitomycin, mitotane, mitoxantrone, mycophenolate, nelarabine, nilotinib, nilutamide, oxaliplatin, paclitaxel, palonosetron, pemetrexed, pentostatin, plicamycin, podofilox, procarbazine, protamine, quinupristin, sirolimus, sorafenib, streptozotocin, sunitinib, tacrolimus, tamoxifen, temozolomide, temsirolimus, teniposide, thalidomide, thioguanine, thiotepa, tinzaparin, topotecan, toremifene, tranexamic acid, urokinase, valrubicin, vinblastine, vincristine, vinorelbine, vorinostat, and warfarin.

Suitable hormones include, but are not limited to, chorionic gonadotropin, conivaptan, corticorelin, corticotropin, cosyntropin, danazol, desmopressin, desogestrel, doxercalciferol, drospirenone, estradiol, estrogen, estrone, estropipate, ethynodiol, etonogestrel, follitropin, glucagon, goserelin, histrelin, lanreotide, leuprolide, levonorgestrel, levothyroxine, liothyronine, lomustine, lutropin, mecasermin, medroxyprogesterone, mestranol, methimazole, nafarelin, norelgestromin, norethindrone, norgestimate, norgestrel, octreotide, oxytocin, paricalcitol, pegvisomant, progesterone, secretin, sermorelin, somatotropin, teriparatide, thyrotropin, triptorelin, and urofollitropin.

Suitable minerals include, but are not limited to, boron, bromine, calcium, chloride, chromium, cobalt, copper, fluoride, iodine, iron, lithium, magnesium, manganese, molybdenum, phosphorus, potassium, rubidium, selenium, sodium, sulfur, zinc, and the various salts thereof, including metal salts, halide salts, organic acid salts, alkoxide salts, carboxylate salts, ammonium salts, and the like.

Suitable neuromodulators include, but are not limited to, acamprosate, acetylcholine, alprazolam, ambenonium, amitriptyline, amoxapine, amphetamine, apomorphine, aripiprazole, armodafinil, atomoxetine, baclofen, benzphetamine, benztropine, biperiden, bromocriptine, bupropion, buspirone, butabarbital, cabergoline, caffeine, carbamazepine, carbidopa, cevimeline, chlordiazepoxide, chlorpromazine, citalopram, clomipramine, clonazepam, clorazepate, clozapine, desipramine, dextromethorphan, dexmethylphenidate, dextroamphetamine, diazepam, diethylpropion, divalproex, donepezil, doxepin, droperidol, duloxetine, edrophonium, ergoloid, escitalopram, estazolam, eszopiclone, ethosuximide, ethotoin, felbamate, flumazenil, fluoxetine, fluphenazine, flurazepam, fosphenytoin, gabapentin, galantamine, haloperidol, imipramine, isocarboxazid, lamotrigine, levetiracetam, levodopa, lisdexamfetamine, lithium, lorazepam, loxapine, maprotiline, memantine, meprobamate, methamphetamine, methsuximide, methylphenidate, mirtazapine, modafinil, molindone, nalmefene, naloxone, naltrexone, nefazodone, nortriptyline, olanzapine, oxazepam, oxcarbazepine, paliperidone, paroxetine, pentobarbital, perphenazine, phendimetrazine, phenelzine, phenobarbital, phentermine, phenyloin, pimozide, pralidoxime, pramipexole, pregabalin, primidone, prochlorperazine, procyclidine, protriptyline, pyridostigmine, quazepam, quetiapine, ramelteon, rasagline, riluzole, risperidone, rivastigmine, ropinirole, rotigotine, secobarbital, selegiline, sertraline, sibutramine, tacrine, temazepam, thioridazine, thiothixene, tiagabine, tizanidine, topiramate, tranylcypromine, trazodone, triazolam, trifluoperazine, trihexyphenidyl, trimethadione, trimipramine, valproate, varenicline, venlafaxine, zaleplon, ziprasidone, zolpidem, and zonisamide.

Suitable nutraceuticals include, but are not limited to, 5-hydroxytryptophan, acidophilus, alpha-linolenic acid, bee pollen, benecol, beta-carotene, beta glucan, brewer's yeast, bromelain, carnitine, catalase, catechins, chalcones, chlorophyll, cholestin, choline, chondroitin, coenzyme Q₁₀, creatine, dehydroepiandrosterone (DHEA), daidzein, dimethyl sulfoxide (DMSO), docosahexanoic acid, eicosapentaenoic acid, flavonoids, genistein, glucomannan, glucosamine, glutamine, glycosides, hesperidin, isoflavones, L-arginine, lactoferrin, lecithin, linoleic acid, lipoic acid, malvin, melatonin, methylsulfonylmethane (MSM), naringin, palmitate, para-aminobenzoic acid, phenylalanine, phosphatidylserine, phytoestrogens, polyphenols, psyllium, proanthocyanidins, pycnogenol, quercetin, resveratrol, rutin, S-adenosylmethionine, sesamol, shark cartilage, silymarin, soy protein, spirulina, ubiquinol, vinpocetine, and xanthine.

Suitable obstetric and fertility agents include, but are not limited to, carboprost, choriogonadotropin, clomifene, dinoprostone, ganirelix, menotropin, methylergonovine, mifepristone, and ritodrine.

Suitable opthalmologic agents include, but are not limited to, apraclonidine, atropine, bimatoprost, brimonidine, brinzolamide, carbachol, cyclopentolate, dipivefrin, dorzolamide, echothiophate, fluorescein, gatifloxacin, homatropine, hyaluronidase, hydroxyamphetamine, hydroxypropyl cellulose, latanoprost, lodoxamide, loteprednol, metipranolol, naphazoline, pegaptanib, pemirolast, pilocarpine, rimexolone, timolol, tropicamide, travoprost, and verteporfin.

Suitable proteins, enzymes and enzyme inhibitors include, but are not limited to, adenosine, albumin, alglucerase, aminocaproic acid, antigens, arginine, cilastatin, clavulanate, cysteamine, cysteine, disulfuram, entacapone, fomepizole, glutamate, glutathione, glycine, glatiramer, imiglucerase, metyrapone, miglustat, nitisinone, pegademase, propylthiouracil, protein fragments, sacrosidase, tazobactam, tolcapone, viruses. polynucleotides,

Suitable respiratory agents include, but are not limited to, acetylcysteine, albuterol, aminophylline, arformoterol tartrate, benzonatate, beractant, budesonide, calfactant, ciclesonide, doxapram, dyphylline, formoterol, guaifenesin, ipratropium, levalbuterol, levonordefrin, menthol, metaproterenol, methacholine, montelukast, nedocromil, oxtriphylline, oxymetazoline, phenylephrine, pirbuterol, poractant, pseudoephedrine, salmeterol, terbutaline, theophylline, tiotropium, zafirlukast, and zileuton.

Suitable rheumatologic agents include, but are not limited to, allopurinol, auranofin, azathioprine, colchicine, leflunomide, penicillamine, probenecid, quinine, and sulfinpyrazone.

Suitable steroids include, but are not limited to, alclometasone, amcinonide, beclomethasone, betamethasone, clocortolone, cortisone, dexamethasone, diflorasone, fludrocortisone, flunisolide, fluocinolone, fluorometholone, fluoxymesterone, fluticasone, halobetasol, methylprednisolone, methyltestosterone, mometasone, nandrolone, oxandrolone, oxymetholone, prednisolone, prednisone, testolactone, testosterone, and triamcinolone.

Suitable urological agents include, but are not limited to, alfuzosin, alprostadil, bethanechol, darifenacin, dutasteride, finasteride, flavoxate, oxybutynin, pentosan polysulfate, propantheline, solifenacin, tamsulosin, terazosin, tiopronin, tolterodine, and trospium.

Suitable vitamins include, but are not limited to, alpha-tocopherol (vitamin E), ascorbic acid (vitamin C), biotin (vitamin B₇), cholecalciferol and calcitriol (vitamin D₃), cyanocobalamin and hydroxocobalamin (vitamin B₁₂), ergocalciferol (vitamin D₂), folic acid (vitamin B₉), inositol (vitamin B₈), menaquinone (vitamin K₂), mendione (vitamin K₃), niacin and nicotinic acid (vitamin B₃), pantothenic acid and pantethine (vitamin B₅), phylloquinone (vitamin Kᵢ), pyridoxine (vitamin B₆), retinol, retinoids, and carotenoids (vitamin A), riboflavin (vitamin B₂), and thiamine (vitamin B₁).

Suitable other agents include, but are not limited to, alcohol, aminohippurate, cinacalcet, citrate, dextrose, dimercaprol, edetate, ferumoxides, ferumoxsil, fludeoxyglucose, gadobenate, gadodiamide, gadopentetate, gadoteridol, gadoversetamide, gallium, glycerin, icodextrin, indocyanine, iodipamide, iodixanol, iohexyl, iopamidol, iopromide, iothalamate, ioversol, ioxaglate, kaolin, lactate, lanolin, lanthanum, mineral oil, pentetate, perflutren, porfimer, petrolatum, samarium, sevelamer, strontium, succimer, technetium, thallous chloride, and trientine.

### Modules not Containing an API

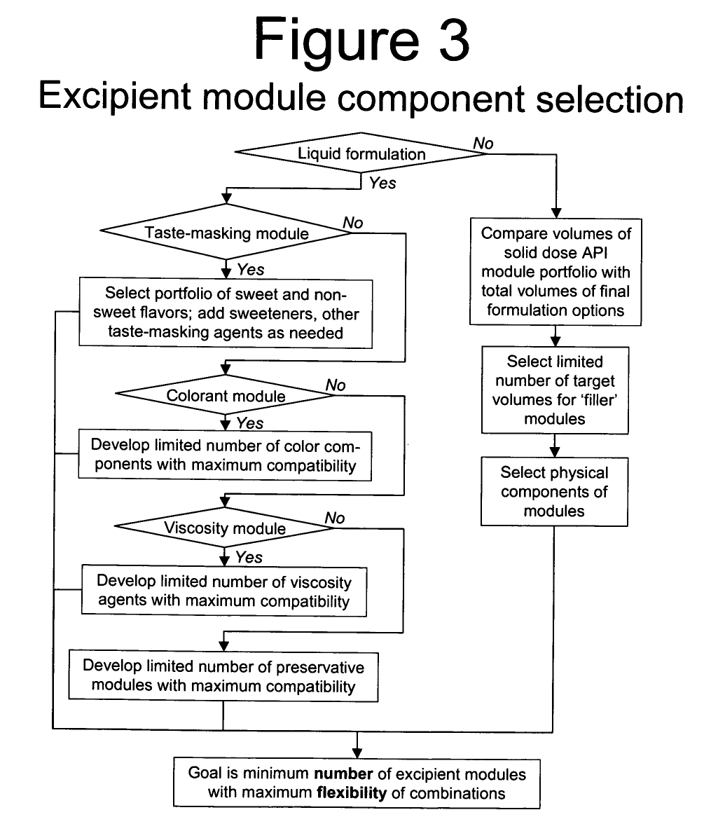

FIG. 3 summarizes the process of developing a portfolio of, and selecting the components in, modules not containing an API, for one embodiment of the present invention. Whereas the key issues that govern the development of precursor modules that contain an API can be relatively straightforward, the key issues that govern the development of precursor modules (especially liquids) that do not contain an API may be complex. To add value, such an excipient-only module may be useful for more than one final product; otherwise it would be simpler to use standard large-volume manufacturing or compounding techniques, for some embodiments. Components of excipient modules may be selected for versatility in combination with the portfolio of API modules, and as the number and type of API modules changes, the portfolio of excipient modules can be optimized. Some excipient modules fall into these classes: fillers used in combination with solid dose API modules; and liquid carriers used in combination with liquid dose API modules, which may contain one or more flavors, sweeteners and other taste-masking agents, colors, viscosity agents, preservatives, and antiseptics.

Ingredients of excipient modules include any suitable ingredients. Such ingredients include, but are not limited to, colorants, flavors, sweeteners and other taste-masking agents, fragrances, and other ingredients used to modify characteristics of the product, including solvents and solutions, diluents, binders, fillers, disintegrants, lubricants, emulsifiers, carriers, suspensions and suspending agents, elixirs, acidifiers, alkalinizers and buffers, solubilizers, surfactants, clarifying, gelling and/or thickening agents, viscosity enhancers, coatings, antioxidants, chelating agents, stabilizers, sustained release agents, antiseptics, preservatives, and multifunction agents.

Suitable colorants include, but are not limited to, dyes and inks approved for use in drugs and cosmetics (D&C), dyes and inks approved for used in food, drugs and cosmetics (FD&C), branded dyes, inks, and coatings, other dyes, and other inks.

Suitable D&C approved colorants include, but are not limited to, D&C Black #1, Blue #1, 2, and 6, Green #1, 4, and 5, Orange #3, Red #3, 4, 5, 6, 7, 19, 21, 22, 27, 28, 30, 33, 36, 39, and 40, Violet #2, and Yellow #5, 6, and 10, including lakes.

Suitable FD&C approved colorants include, but are not limited to, FD&C Blue #1, 2, 10, 40, Green #1 and 3, Orange #2, Red #1, 2, 3, 4, 7, 19, 27, 28, 30, 33, and 40, Violet #1, and Yellow #1, 3, 5, 6, and 10, including lakes.

Suitable branded colorants include, but are not limited to, BLUE TEK PRINT SB-6008 and SB-6029; CHROMA-TERIC Deb-5037-Ore, T3000-We, and Yellow T3277-Ye; CHROMA-TONE PDDB-8906-W and PDDB-8746-Or; CHROMACOTE T 2700GN, T 2716Y, and T 2956Y; COATERIC YPA-6-7430 White and YPA-6-7089 White; DIOLACK 00f32892 Yellow; DRI KLEAR; DRI KLEAR 042; DRI KLEAR LV 609527; DRY FLO; DRY-CLEAR LV; DURO-TAK 80-1196, 87-2070, 87-2194, 87-2287, 87-2296, 87-2888, 87-2979, and 280-2516; FLEXOGRAPHIC Pink; PHARMACOAT 606; Red and White COTOLENE-P; SPECTRABLEND Csl-15764 (Blue); SPECTRASPRAY Blue 50726; WHITE TEK PRINT SB-007p.

Additional branded colorants and coatings include, but are not limited to, OPACOAT Na2203, Na4108 Blue, Na4711 Lavender, Na7013 Clear; OPACODE A-10450 Black, A-10509 Black, Ns-78-10013-N, Ns-78-17502 Gray, Ns-78-17821, Ns-78-8000 Black, Ns-78-8001, Nsp-78-17734 Black, S-1-13001 Orange, S-1-15034-Fd Red, S-1-15038 Red, S-1-16507, S-1-1666 Red, S-1-1666-M Red, S-1-1681 Red, S-1-17706 Black, S-1-17711 Black, S-1-17734 Black, S-1-17749 Black, S-1-17762 Black, S-1-17797 Black, S-1-18025 White, S-1-26514 Brown, S-1-3110 Green, S-1-3171 Green, S-1-4124 Blue, S-1-4157, S-1-4159 Black, S-1-4160 Blue, S-1-4172 Blue, S-1-4172m Blue, S-1-4362, S-1-7020, S-1-7077, S-1-7078, S-1-7085 White, S-1-7090 White, S-1-7534 Gray, S-1-800hv Black, S-1-8025 Black, S-1-8081 Black, S-1-8090 Black, S-1-8092 Black, S-1-8093 Black, S-1-8094 Black, S-1-8095, S-1-8100-Hv Black, S-1-8105 Black, S-1-8106 Black, S-1-8109 Black, S-1-8110 Black, S-1-8114 Black, S-1-8115 Black, S-1-8152hv Black, S-1-9009 Brown, S-1-9032, S-1-9037 Brown, S-1-9048c, S-1-9460hv Brown, S-19-7014 White, S-8-20931, Sb-4028 Green, Wb Ns-78-10521 Blue, Wb Ns-78-17715 Black, Wb Ns-78-18001 White, and Wb Nsp-78-18022 White.

Also, OPADRY 00a28646, 02b14941 Pink, 02b22429 Yellow, 02b94016 Pink, 02g22555 Yellow, 02g24523 Pink, 02g26637 Brown, 02g28619 White, 02-H-22703 Yellow, 03a 58900 White, 03a14309 Pink, 03b11434 Green, 03b12878 Yellow, 03b12896 Yellow, 03b12914 Yellow, 03b14424 Pink, 03b14436 Pink, 03b16083 Maroon, 03b17426 Beige, 03b17495 Beige, 03b22426 Yellow, 03b24562 Peach, 03b50899 Blue, 03b54504 Pink, 03b54573 Pink, 03b54588 Pink, 03b54955 Pink, 03b56518 Brown, 03b56982 Brown, 03b57631 Grey, 03b58902 White, 03b58930 White, 03b58965 White, 03b86625 Brown, 03b86636 Brown, 03f12920 Yellow, 03f12967 Yellow, 03f13325 Orange, 03f14895 Pink, 03f54568 Pink, 03j18312 White, 03k14881 Pink, 03k50891 Blue, 03k51211 Green, 03k52543 Yellow, 03k54121 Pink, 04f50702 Blue, 04f58804 White, 05b10446 Purple, 05b10457 Purple, 05b11552 Green, 05b11781 Green, 05b12337 Yellow, 05b15325 Red, 05b17055 Tan, 12b58900 White, 12f20984 Blue, 12f21129 Green, 12f22609 Yellow, 12j18255 White, 13b50780 Blue, 13b51260 Green, 13b52329 Yellow, 13b58802 White, 13h52750 Yellow, 13h52754 Yellow, 13h54756 Pink, 13h54757 Pink, 15b111947 Green, 15b13335 Orange, 15b20780 Blue, 15b21340 Green, 15b22275 Yellow, 15b24473 Pink, 15b24879 Pink, 15b28665 White, 15b53449 Orange, 16b38982 White, 16b5900 Yellow, 20014832 Pink, 20a52229 Yellow, 20a52560 Yellow, 20a52900 Yellow, 20a54211 Pink, 20a54239 Pink, 20a54614 Pink, 20a54616 Pink, 20a54900 Pink, 20a54901 Pink, 20a56500 Brown, 20a56694 Brown, 20a56788 Brown, 20a58806 White, 20a58916 White, 20a59015 Clear, 20b11521 Green, 20b17583 Gray, 20b97160 Beige, 20c15347 Red, 20h52619 Yellow, 20h58983 White, 31f20963 Blue, 31f32870 Yellow, 32k14834 Pink, 32k23123 Orange, 33g12976 Yellow, 33g25171 Brick Red, 40114278 Pink, 80w 12319 Yellow, 80w22657 Amb Yellow, 80w-93032 Amb Orange, 85f14999 Pink, 85g93096 Orange, Amb 80w52110 Yellow, I 03b22409 Yellow, I 03b23197 Orange, and I 03b24658 Pink.

Also, OPADRY II 03b10903 Blue, 31f22071 Yellow, 31f22088 Yellow, 31f23111 Orange, 31f24239 Pink, 31f27625 Gray, 31f32090 Yellow, 31f58914 White, 31k52633 Yellow, 32b10817 Blue, 32k10054 Purple, 32k12160 Yellow, 32k12884 Yellow, 32k12942 Yellow, 32k12968 Yellow, 32k13357 Orange, 32k13699 Orange, 32k14826 Pink, 32k14833 Pink, 32k16706 Brown, 32k17089 Tan, 32k17573 Gray, 33g10907 Blue, 33g11635 Green, 33g28707 White, 40 L14235 Pink, 40 L17589 Gray, 40014876 Pink, 40b12994 Beige, 40b97172 Yellow, 40c10881 Blue, 40c13396 Orange, 40c18303 White, 40l10412 Purple, 40l10884 Blue, 40l11438 Green, 40l11588 Green, 40l12917 Yellow, 40l12979 Yellow, 40l13950 Orange, 40l14190 Pink, 40l14336 Pink, 40l14836 Pink, 40l17427 Beige, 40l17587 Gray, 40l92058 Yellow, 40o93122 Orange, 49b10882 Blue, 49b13460 Orange, 49b16716 Brown, 85f10919 Blue, 85f12345 Yellow, 85f12372 Yellow, 85f13980 Orange, 85f16876 Brown, 85f18378 White, 85f18422 White, 85f22055 Yellow, 85f23470 Pink, 85f24033 Pink, 85f24307 Pink, 85f28751 White, 85f288751 White, 85f94172 Pink, 85g20583 Blue, Oy-L-22903, Oy-L-23028 Orange, Oy-L-24802 Pink, Oy-L-24803 Pink, Oy-L-24808, Oy-L-32920, Pink 85g94027, Pink 85g94065, Red 85g94101, Y-19-19054 Clear, Y-19-7483 Clear, Y-22-10274 Lavender, Y-22-10508 Blue, Y-22-10519 Blue, Y-22-10538 Blue, Y-22-10702 Blue, Y-22-10764 Blue, Y-22-11184 Green, Y-22-11210 Green, Y-22-11251 Green, Y-22-12098 Yellow, Y-22-12664 Yellow, Y-22-12718 Yellow, Y2212720 Pale Yellow, Y-22-12780 Yellow, Y-22-13034 Orange, Y-22-13061 Orange, Y-22-13083 Orange, Y-22-13089 Orange, Y-22-13167 Orange, Y-22-13526 Orange, Y-22-13577 Flesh, Y-22-13603 Orange, Y-22-13613 Orange, Y-22-13663 Orange, Y-22-14001 Pink, Y2214701 Pink, Y-22-15061, Y-22-16562 Brown, Y-22-16577 Brown, Y-22-17025 Beige, Y-22-17165 Beige, Y-22-17221 Beige, Y2217279 Beige, Y-22-17515 Gray, Y-22-18238 White, Y-22-7719 White, Y-30-10671a Blue, Y-30-10701 Blue, Y-30-12705 Yellow, Y-30-12736a Yellow, Y-30-12737a Yellow, Y-30-12842a Yellow, Y-30-12863a Yellow, Y-30-13091 Orange, Y-30-13242a Orange, Y-30-13616 Orange, Y-30-13642a Orange, Y-30-14700a Pink, Y-30-14758 Pink, Y-30-17295a Tan, Y-30-17296a Beige, Y-30-17340a Beige, Y-30-17528 Gray, Y-30-18037 White, Ys-1-12524a, Ys-1-19025a Clear, Ys-1-7006 Clear, Y—S-1-7006 Clear, Ys-22-13571 Orange, Ys-22-17227a Beige, Ys-22-18096 White, Ys-30-12788a Yellow, Ys-30-13641a Orange, Ys-30-14743a Pink, Ys-30-14777a Pink, Ys-30-17265a Beige, Ys-30-17271a Beige, and Ys-30-18105 White.

Also, OPADRY 02b24864 Pink; Opadry OS-F-32867 Yellow; Opadry OY-22959 Yellow, 23050 Orange, 27202 Tan, 27301 Butterscotch, 3736 Butterscotch, 38924 White, 52945 Yellow, 54937 Pink, 58900 White, 7240 Clear, 7300 White, 8764h Orange, B-28920 White, B-32830, Gm-28900, L-27204 Tan, L-27205 Beige, L-28906, L-34836 Pink, Ls-20921 Blue, Ls-23016 Orange, Ls-23018 Orange, Ls-28908 White, Ls-28914 White, Ls-33111 Orange, Ls-37200 Buff, Ls-58900 White, S-1387 Pink, S-20007 Purple, S-20900 Blue, S-20901 Blue, S-21001 Green, S-21027 Green, S-22802 Yellow, S-22815 Yellow, S-22907 Yellow, S-23049 Orange, S-24900 Pink, S-24972 Pink, S-26529 Red, S-26530 Red, S-28833 White, S-28849 White, S-28924 White, S-29019 Clear, S-30013 Purple, S-30913 Blue, S-30953 Blue, S-32921 Yellow, S-32986 Yellow, S-33016, S-34800 Pink, S-34817 Pink, S-34923 Pink, S-34995 Pink, S-38928, S-38944 White, S-52902 Yellow, S-53010 Orange, S-54902 Pink, S-54904 Pink, S-6937 Pink, S-7322 White, S-7399 White, S-9476 Brown, S-9603 White, and Sr-34907.

Also, OPADRY S-1-1666 Red; Opadry Y-1-1518 Pink; Opadry Y-1-17272a Beige, 1-17517 Gray, 1-2102 Yellow, 1-2132 Yellow, 1-2516 Orange, 1-2553 Orange, 1-2605 Beige, 1-3211 Green, 1-4205 Blue, 1-4206 Blue, 1-4234 Blue, 1-7000 White, 1-7000b White, 1-7000h White, 1-7006 Blue, 1-7503 Gray, 22-10501 Blue, 22-12720 Pale Yellow, 22-12751 Yellow, 22-13558 Orange, 22-14525 Pink, 22-15008 Red, 22-15119 Red, 22-18238 White, 30-13168a Orange, 30-14565 Pink, 30-17267 Beige, 33g-27241 Beige, 5-10272a Lavender, 5-10300 Lavender, 5-10670 Blue, 5-1244 Pink, 5-12539 Yellow, 5-12544a Yellow, 5-12584 Yellow, 5-13512 Orange, 5-13513 Orange, 5-14530a Pink, 5-1727 Red, 5-2028 Yellow, 5-2042 Yellow, 5-2086 Yellow, 5-2312 Yellow, 5-2328 Orange, 5-2371 Orange, 5-2394 Orange, 5-2450 Orange, 5-2451 Orange, 5-2644 Beige, 5-2646 Beige, 5-3140 Green, 5-3171 Green, 5-3193 Green, 5-3296 Green, 5-4129 Blue, 5-4270 Blue, 5-4287 Blue, 5-4295 Blue, 5-6233 Light Orange, 5-6301 Yellow, 5-6308, 5-7058 White, 5-7068 White, 5-7072 White, 5-7411 Purple, 5-7524 Grey, 5-8050 Black, 5-9006 Brown, and 5-9020 Brown.

Also, OPADRY Yellow; OPADRY YPS-7-2127; OPADRY YS-1-003 White, 1-10010 Purple, 1-10291 Lavender, 1-10523a Blue, 1-10525 Blue, 1-10533a, 1-10542a Blue, 1-10547a Blue, 1-10563 Blue, 1-10613a Blue, 1-10618, 1-10629, 1-10654a Blue, 1-10682 Blue, 1-10690a Blue, 1-10699 Blue, 1-10745 Blue, 1-10748a Light Blue, 1-10755 Blue, 1-10783a Blue, 1-11000 Pink, 1-11051 Green, 1-11060 Green, 1-1107 Green, 1-11075a Green, 1-11113 Green, 1-11171 Green, 1-11234 Green, 1-11305 Green, 1-11369 Green, 1-1246 Pink, 1-1252 Pink, 1-12524a Yellow, 1-12525a Yellow, 1-12526a Yellow, 1-12529 Yellow, 1-12541 Yellow, 1-1256-A Yellow, 1-12573 Yellow, 1-12581 Yellow, 1-1262 Pink, 1-12625 Yellow, 1-12702a Yellow, 1-12732 Yellow, 1-1277 Pink, 1-12789 Yellow, 1-12826 Yellow, 1-1283 Pink, 1-12844 Yellow, 1-12847 Yellow, 1-1298 Pink, 1-13013 Peach, 1-13065a Orange, 1-13119 Orange, 1-13121 Yellow, 1-13148a Orange, 1-13214 Orange, 1-13269 Orange, 1-13271 Orange, 1-13555 Orange, 1-13591a Orange, 1-13664a Orange, 1-13673a Orange, 1-13675a Orange, 1-14012 Pink, 1-14129 Pink, 1-14130 Pink, 1-1418 Pink, 1-1441g, 1-1448g Pink, 1-14518a Pink, 1-14519a Pink, 1-14532 Pink, 1-1454 Pink, 1-14555a Pink, 1-14559 Pink, 1-1456g Pink, 1-14587a Pink, 1-14593a Pink, 1-14595 Pink, 1-14608a, 1-14643a Pink, 1-14725 Pink, 1-14756a Pink, 1-14779a Pink, 1-15050 Red, 1-1510 Pink, 1-1528 Pink, 1-1543 Pink, 1-15585a Red, 1-16002 Maroon, 1-16518a Brown, 1-17169a, 1-17180a Beige, 1-17181a Beige, 1-17192a, 1-17209 Beige, 1-17220, 1-17222a Tan, 1-17235a Peach, 1-1724 Red, 1-17274a Beige, 1-17277a Beige, 1-17307a Butterscotch, 1-17505a Gray, 1-17506a Gray, 1-1751g Red, 1-1755 Gray, 1-18005 White, 1-18022 White, 1-18027 White, 1-18027a White, 1-18028 White, 1-18097a White, 1-1811 Red, 1-18111 White, 1-18130a White, 1-1814 Red, 1-18177a White, 1-18202a White, 1-18229 White, 1-1846 Red, 1-1847 Red, 1-1891 Red, 1-19025-A Clear, 1-2007 Yellow, 1-2013 Yellow, 1-2053 Yellow, 1-2063 Yellow, 1-2074 Yellow, 1-2083 Yellow, 1-2115 Yellow, 1-2122 Yellow, 1-2134 Yellow, 1-2135 Yellow, 1-2136 Yellow, 1-2141 Yellow, 1-2152 Yellow, 1-2167 Yellow, 1-2181 Yellow, 1-2184 Gold, 1-2186 Yellow, 1-2192 Yellow, 1-2305 Orange, 1-2308 Dark Orange, 1-2344 Yellow, 1-2383 Orange, 1-2398 Orange, 1-2449 Orange, 1-2455 Red, 1-2465, 1-2487 Orange, 1-2522 Orange, 1-2526 Orange, 1-2527 Orange, 1-2534, 1-2546 Orange, 1-2548 Orange, 1-2549 Orange, 1-2558 Orange, 1-2563 Orange, 1-2564, 1-2578 Orange, 1-2596 Orange, 1-2604 Beige, 1-2612 Beige, 1-2619, 1-2621 Rust, 1-2623 Brown, 12630 Yellow, 1-2635 Tan, 1-2660 Salmon, 1-2665 Beige, 1-2669 Rust, 1-2671 Beige, 1-3105 Green, 1-3130 Green, 1-3134 Green, 1-3146 Green, 1-3147, 1-3166 Green, 1-3256 Green, 1-3288 Green, 1-4014 Blue, 1-4018 Blue, 1-4112 Blue, 1-4137 Blue, 1-4215, 1-4216, 1-4221 Blue, 1-4228 Blue, 1-4229 Blue, 1-4234 Blue, 1-4235 Blue, 1-4236 Blue, 1-4240 Blue, 1-4241 Blue, 1-4245 Blue, 1-4249 Blue, 1-4251 Blue, 1-4254, 1-4255, 1-4256 Blue, 1-4282 Blue, 1-4298 Blue, 14644 Pink, 1-4700 Purple, 1-4710, 1-4739 Lavender, 1-4812 Lavender, 1-4845 Purple, 1-6275 Orange, 1-6300, 1-6312 Yellow, 1-6318 Yellow, 1-6320 Yellow, 1-6357 Yellow, 1-6370g Yellow, 1-6378g Yellow, 1-6381 Yellow, 1-6382g Yellow, 1-7000e White, 1-7002 White, 1-7003 White, 1-7003h White, 1-7006 Clear, 1-7022 Off-White, 1-7027 White, 1-7040 White, 1-7052 White, 1-7059 White, 1-7060 White, 1-7086 White, 1-7191 Brown, 1-7444g White, 1-7449 White, 1-7472 Clear, 1-7507 Grey, 1-7552 Grey, 1-7700 White, 1-7706g White, 1-8325 Beige, 1-8343g Beige, 1-8345g Beige, 1-8608 Orange, 1-8619 Orange, 1-89193 Clear, 1-9011 Brown, 1-9012 Brown, 1 r1418 Pink, 1 r-7006 Clear, 2-10657 Blue, 2-19071a Clear, 2-19114a Clear, 22-16576 Brown, 22-18119 White, 2-7013 Clear, 2-7063 White, 3-7011 Clear, 3-7031 Clear, 3-7413 Clear, 5-12575 Yellow, 5-12576 Yellow, 5-1260 Pink, 5-1296 Pink, 5-17266 Tan, 5-18068 White, 5-18074 White, 5-2085 Yellow, 5-2370 Orange, 5-3116 Green, 5-4277 Blue, 5-4278 Blue, 5-7017, 5-7042 Clear, 5-7068, and 5-7099 White.

Also, OPAGLOS GS 2-0300 and 2-0310; OPAGLOS S 0750; OPALUX AS 1406 Pink, 1475 Pink, 1537 Pink, 1589 Pink, 2006 Yellow, 2007 Yellow, 2052 Yellow, 2062 Yellow, 2086 Chartreuse, 2094, 2167 Yellow, 2236, 2269 Yellow, 2324 Orange, 2336 Orange, 2395 Peach, 2413, 2433 Orange, 2490 Coral, 2498 Orange, 2553 Orange, 2612, 2613 Tan, 2620-B Tan, 2676 Salmon Jasper Red, 2754, 2768, 2787 Butterscotch, 3140 Green, 3287, 3288 Green, 3308 Green, 3348-C Green, 3376, 3381, 3389 Green, 3391 Green, 3942 Maroon, 4025, 4151 Blue, 4188 Blue, 4193 Blue, 4208-A Blue, 4258 Blue, 4270 Blue, 4800 Lavender, 4854 Lavender, 4855 Purple, 4891, 5034 Red, 5107, 5162 Green, 5178 Green, 5203 Green, 5212 Green, 7000-B, 7000-P White, 7001, 7535 Gray, 8010-A Black, 8050-L Black, 9010 Brown, 9030 Brown, and 9050 Brown.

Also, OPALUX Blue and Green; Opaque Blue 100, Blue 147, Blue 605, Brown 85 Bfj, Green 1664, Green 97, Green/Flesh, Maroon 6 Dar, Pink 0439, White 001, White 002, White 535, White 536, White 538, and White 8.

Also, OPASPRAY 3-1700, 3-1810, 3-1820, 3-1830, Im-176, K-1-1230 Pink, K-1-1243, K-1-1254, K-1-1279, K-1-1289 Pink, K-1-14016 Pink, K-1-1413 Pink, K-1-1414 Pink, K-1-1432, K-1-1437, K-1-1455 Pink, K-1-1526 Pink, K-1-1563 Pink, K-1-1573 Lavender, K-1-1574, K-1-1584, K-1-1719 Red, K-1-2004 Yellow, K-1-2013 Yellow, K-1-2043 Yellow, K-1-2182 Yellow, K-1-2186 Yellow, K-1-2216-A Yellow, K-1-2227 Yellow, K-1-2228 Yellow, K-1-2239, K-1-2240 Yellow, K-1-2256 Yellow, K-1-2275 Yellow, K-1-2300 Peach, K-1-2301 Peach, K-1-2303 Orange, K-1-2304 Orange, K-1-2314 Orange, K-1-2327 Orange, K-1-2330 Orange, K-1-2335 Orange, K-1-2406 Orange, K-1-2410 Orange, K-1-2417 Orange, K-1-2430, K-1-2441 Orange, K-1-2471 Orange, K-1-2473, K-1-2492, K-1-2531, K-1-2533 Orange, K-1-2554, K-1-2568 Orange, K-1-2570 Orange, K-1-2588 Orange, K-1-2614 Beige, K-1-2621 Brown, K-1-2626 Orange, K-1-2656 Beige, K-1-2670 Tan, K-1-2674 Beige, K-1-2685, K-1-2711, K-1-2723 Butterscotch, K-1-2837, K-1-3000, K-1-3142 Green, K-1-3144 Green, K-1-3147, K-1-3148 Green, K-1-3156, K-1-3173 Green, K-1-3178 Green, K-1-3197 Green, K-1-3202 Green, K-1-3209 Green, K-1-3220 Green, K-1-3227, K-1-3300-A Green, K-1-3300-C Green, K-1-4108 Blue, K-1-4119, K-1-4122 Blue, K-1-4136 Blue, K-1-4205 Blue, K-1-4210-A, K-1-4213 Blue, K-1-4214, K-1-4227, K-1-4234 Blue, K-1-4235 Blue, K-1-4728, K-1-4731 Purple, K-1-4743 Lavender, K-1-4786, K-1-5024 Red, K-1-7000 White, K-1-70008 White, K-1-7000b, K-1-7076, K-1-9027 Brown, K-1-9039-L Brown, K-1-9060 Red, K-1-9080 Brown, K-1-9112 Brown, L-2113, L-3305 Green, L-3306 Green, L-7000 White, M-1-2042, M-1-3459 B Orange, M-1-4395b Blue, M-1-7111-B, M-1-711b White, M-1-7120 White, M-1-8429 Yellow, and Wd-1270 Pink; OPATINT Ad-25000 Red, Dd-13009 Orange, Dd-14000 Pink, Dd-1800 White, and Dd-18000 White.

Suitable other dyes include, but are not limited to, Beige P-1437; Black LB-260, 442, 636, 1171, and 9972; Black Oxide; Blue LB-332, 781, and 1245; Blue Lakolene; Brown LB-292, 464, 1685, 1792, and 56069; Burnt Umber; Caramel 105; Caramel Acid Proof 100; Carmine 09349; Casing 27-75; Emerald Green LB-9207; Ferric Oxide Brown, Green, Orange, Pink, Red, Red-Brown, and Yellow; Gray #2982; Green 70363; Green LB-265, 279, 333, 482, 555, 603, 820, 883, 1174, 1236, 1441, 1594, 1616, 1644, 3323, and 9583; Green PB-1543 and 1766; Green PMS-579; Green PR-1333 and 1339; Lavender LB-1356 and 1603; Mint Green; Ochre 3506; Orange 54172; Orange LB-452, 715, 1387, 1439, and 3810; Peach LB-1576; Pink; Purple LB-562, 588, 639, 694, and 1902; Red LB-6053-R and 9570; Red PB-1595; Salmon LB-1668; Swedish Orange #2191; Tan PB-1388; Tetrarome Orange; Turquoise LB-1430; White TC-1032; Wild Cherry 7598; Yellow LB-104, 111, 1577, 1637, 1769, and 9706; Yellow PB-1345 and 1381; Yellow WD-2014; Yellow #70362; and Yellow Ochre.

Suitable other inks include, but are not limited to, Black 2271, A-10464, A-10527, FGE-1386, GG-606, S-1-8100-Hv, SW-9007, SW-9008, SW-9009, and SW-9010; Blue and Yellow Imprint GG-823; Blue Black A-10463, and A-9371; Blue S-1-10551 and S-1-4118; Dark Yellow and Yellow Imprint GG-824; Edible Black, Blue, Brown, Gray, Orange, Pink, Red, Red A-8032, and White; Fine Black 2202c and 2212; Green A-10454 and A-10629; Orange and Yellow Imprint GG-822; Pink Imprinting SB-1003; Red 5-1-9034 and A-8032; Red and Aqua Imprinting GG-827; Red and Caramel Imprinting Gg-825; Red Imprinting Gg-826; S-1-7085; Thinner; and White 21-K, A-8154, S-1-7075, and SB-0007p.

Suitable flavors, sweeteners, and taste-masking agents include, but are not limited to, Anise 29653; Apple Watermelon PFC 9887; Apricot 23067 and 24829; Apricot Peach; Banana 15223, 501013 AP0551, 71507, 74546, FMC 23406, and SA84; BBA-47769; Berry Citrus Blend 8409, 9621, and 9756; Bitter Mask 9885; Bitterness Modifier 15555, 36734, and 367343; Black Cherry 501027 AP0551; Blood Orange 51.226T and SA; Bubble Gum 15864, 175303, 3266P, and MC-4938; Buttermint 24020; Butterscotch 61005-U and F-1785; Candied Sugar 510155U; Cherry 57.679/A, 213, 349, 594 S.D., 825.476WC, 842, 1566, 3321, 8513, 11539, 11929, 104613, 107026, 181612, 231494, 338614, 500910U, 590271A, 598384, A-22872, DP300684, EP-3699, F-232, FI-8568, FMC 22872, FMC 8513, IFF 13530912, L-1233, N-2755, NV-1489, PFC-9768, R-6556, WL-1093, WL-18022, and WL-4658; Cherry Berry F-1194; Cherry Blend 770; Cherry Burgundy 11650; Cherry Cream 14850; Cherry Maraschino S-3531, Cherry Mint 5073A; Cherry Pistachio PFC-8450; Cherry Vanilla Compound A77487; Cherry-Anise PFC-9758; Chloroform 103589; Chocolate P727; Coconut 41 and F-3893; Coconut Toasted 1323PG; Cola FMC 15740; Cotton Candy 30-92-0011 and F-9967; Cough Syrup 819, 110257, and 134681; Cream EP-17688; Cream Soda; Creamy Vanilla 16345; Creme 46971; Creme de Menthe 14677; Creme de Vanilla 28156; Curacao 50.397A; and Custard 52.940/A FIR.

Also, DF-119, DF-1530, and E-472; Enhancer F-5397A and F-9843; FIG. 827118; Fritzsche 21028-D, 46215, 73959, 75021, and 78087; Fruit 01-10428 and 84.6422; Fruit Gum 912; Fruit Mint 75588; Fruit Punch #716, #28140, and 14761 FM; Grape 59.145/APO5.51, 59.266/APO5.51, 6175, 054158, 486939, 501040A, 13403873, IFF 13549478, PFC 8439, PFC-9711, and PFC-9924; Guarana FMC-15417; IFF FP 69 F; Kiwi S-718; Lemon 812 and FMC-10471; Lemon Mint 862.547; Lemon Mint Fritzsche 54369; Lemon Spray V3938-1 N1; Mandarin 15228-71; Maque Tree 377(BUSH); Mask RBT-NV-7759; Masking 35321; Menthol Mint PFC-9926; Mint 287, 51296 TP0551, and SN027513; Mixed Fruit PFC-9970; and NNS DZ-03226.

Also, Orange #7679, 9/79J839, 57.458/AP05.51, 739K (PB82), 13334, 74016-71, 249792, 607217, G-10431, P-5614, and PFW-730016U; Orange Banana WL-18093; Orange Pineapple FV-43; Peach 10457, 302789, and 13503584; Peach Mint Fritzsche 106109; Peach Pineapple FMC 14258; Peppermint 104, 517, 894.143, K373, PFC 9927, and WL-6167; Peppermint Stick FMC 16170; Pineapple 182661, N-2566, and N-2766; Pineapple-Coconut, Primary Taste Modifier # 29275; Punch 610962U and WL-7126; Raspberry 954, 954K (BK77), 998, 1840, 8456, 21028D, 28106, 50776, 65934, 262085, A11693, D9599, DY-04447, F-1784, F-1840, F-6887-S, and PFC-8407; and Root Beer 180339.

Also, Strawberry 052311 AP0551, 133.5655, 17.36.8509, 17C56217, 52.311AP, 5210(FD&D), 52312/AP, 5951, 9843, 14953, 55058, 523121A, DY-04359, F-5665, F-5930-A, FN-13819, PFC-9626, and WL-16650; Strawberry Guarana 586.997/APO5.51; Sweet 24052 and 604978; Sweet Tone 28837 and AM 918.005; Sweetness Enhancer 5401 B; Tangerine Fritzsche 51465; TM 313298; TPF 135 and TPF 143; Tropical Blend FV-50; Tropical Fruit Punch 1591 and N&A 50432; Tutti Frutti 0002028, 24093FM, 51.880/AP05.51, P-5400, and WL-18481; Vanilla 323453, 33869, 501441 AP2004, F-6257, P-1160, PFC-8541, and PFC-9772; Wild Cherry 29653, 695047U, K-321, NV-101-1489, PFC-14783, and WL-1093; Wintergreen PFC-8421, Yellow Plum Lemon 39K 020, High Fructose Corn Syrup; and Flavors #18317, 57000 IU, 57820/A, 89-186, and 89-259, and other sweeteners and taste-masking agents.

Suitable branded flavors include, but are not limited to, AROMALOK 182608 and 262453; Banana DURAROME 860.095 TD09.91; C&K Mixed Fruit A13688; Cheri Beri PCD-5580, PFC-8573, PFC-8580, and PFC-8573; Cherry DURAROME 860.097 TD10.91; Cherry E.P. Modified 151; Cherry FONA 825.662; Cherry H&R PHARMA 004; Cherry WIXON 3566; FELTON 6-R-9; FLORASYNTH; Fruit Tak 20008; Grape FIRMENICH 587.444 and 597.303/C; Grape GIVAUDAN 433160; Grape MANHEIMER 522463; Grape MICRON ZD-3876; Grape Nector PFC-8599; HAVERSTROO ZD 49284; Lemon GIVAUDAN 74940-74; MAGNASWEET 100, 110, 135, 180, and 185; MCP Lemon DURAMONE 4409A; MCP Lime DURAMONE 6419; Menthol VERALOCK; Orange GIVAUDAN 74388-74; Orange WONF 608352; Orbit Serene 20340; Peppermint SEELOCK 34907; PERLAROM Strawberry; Pharmaceutical 182608; PHARMASWEET 10772900; PLUSWEET; PROSWEET 604, 694, and K; Raspberry AROME PFC-9908; Raspberry POLAK 5000064; Refrachessment FD-8027D; RHODIA PHARMACEUTICAL #RF 451; Strawberry MICROSEAL; Strawberry TRUSIL WINDSOR 237303; TETRAROME; Tutti Frutti PERMASEAL 77919-31; Vanilla BECK C7984; VERALOCK Bubble Gum; and Wild Cherry GIVAUDAN F-1813.

Suitable fragrances include, but are not limited to, Essence Bouquet 9200, Fritzbro Orange, Lemon, and Orange; Fragrance 3949-5, 520a, 6.007, 91-122, 9128-Y, 93498g, Balsam Pine #5124, Bouquet 10328, CHEMODERM 6401-B and 6411, Cream #73457, Cs-28197, Felton 066m, FIRMENICH 47373, GIVAUDAN Ess 9090/Ic, H-6540, Herbal 10396, Nj-1085, P 0 Fl-147, Pa 52805, PERA DERM D, Rbd-9819, SHAW Mudge U-7776, Tf 044078, UNGERER Honeysuckle K 2771, and UNGERER N5195; and Perfume 25677, Bouquet, E-1991, Gd 5604, TANA 90/42 Scba, and W-1952-1.

Suitable other excipients include, but are not limited to, 1,1,1-trichloroethane; 1,1,2,2-tetrafluoroethane; 1,2,6-hexanetriol; 1,2-dimyristoyl-sn-glycero-3-(phospho-s-(1-glycerol)); 1,2-dioleoyl-sn-glycero-3-phosphocholine; 1,2-dipalmitoyl-sn-glycero-3-(phospho-rac-(1-glycerol)); 1,2-distearoyl-sn-glycero-3-(phospho-rac-(1-glycerol)); 1,3-dimethylol-5,5-dimethyl-hydantoin; 1-aminocyclohexanecarboxylic acid, c-11; 1-methyl-2-pyrrolidinone; 1-o-tolylbiguanide; 2-amino-2-methyl-1-propanol; 2-ethylhexyl salicylate; and 2-naptholene sulfonate sodium salt.

Also, acacia; acesulfame potassium; acetic acid; acetic anhydride; acetone; acetone sodium bisulfite; acetophenone; acetylated monoglycerides; acetylcysteine; acetyltributyl citrate; acetyltryptophan; acrylates copolymer; acrylic acid; activated charcoal; adipic acid; AEROTEX resin 3730; agar; air; alanine; albumin; alcohol; alfadex; alginic acid; alkyl ammonium sulfonic acid betaine; alkyl aryl sodium sulfonate; allantoin; almond oil; alpha-terpineol; alpha-tocopherol; and althea.

Also, aluminum acetate, chlorhydroxy allantoinate, diacetate, hydroxide monostearate, oxide, polyester, potassium sulfate, silicate, silicate pentahydrate, starch octenylsuccinate, stearate, and sulfate, and other aluminum compounds.

Also, ALZAMER-39 and 50; AMBERLITE; AMERCHOL c and cab; ammonia; and ammonio methacrylate copolymer.

Also, ammonium acetate, calcium alginate, chloride, glycyrrhizate, hydroxide, lauryl sulfate, nonoxynol 4 sulfate, phosphate, and sulfate, and other ammonium compounds.

Also, ammonyx; amphoteric-2; amphoteric-9; amyl acetate; anethole; anidrisorb 85/70; anise; anoxid sbn; antifoam; antipyrine; apricot kernel oil peg-6 esters; aquacel 126; aquacoat; aquaphor; arginine; arlacel; arlatone 289; ascorbic acid; ascorbyl palmitate; aspartame; aspartic acid; attapulgite; balsam; barium sulfate; b-cyclodextrin sulfobutyl ether sodium; beeswax; beheneth-10; bentonite; benzaldehyde; benzalkonium chloride; benzenesulfonic acid solution; benzethonium chloride; benzododecinium bromide; benzoic acid; benzoin; benzyl alcohol, benzoate, and chloride; betadex; beta-naphthol; betose; bibapcitide; bismuth subcarbonate and subgallate; boric acid; brocrinat; butane; butyl alcohol, ester, and stearate; butylated hydroxyanisole and hydroxytoluene; butylene glycol; butylparaben; c20-40 pareth-24; and caffeine.

Also, calcium acetate, alginate, ascorbate, carbonate, carrageenan, chloride, citrate, cyclamate, gluceptate, hydroxide, lactate, phosphate, pyrophosphate, salicylate, stearate, sulfate, sulfate dihydrate, and sulfate hemihydrate, and other calcium compounds.

Also, caldiamide sodium; calteridol calcium; candelilla wax; canola oil; caprylic triglyceride; capsicum oleoresin; captan; and caramel.

Also, carbomer 934, 934p, 940, 941, 974, 974p, 980, 981, and 1342; carbon; carbon black; carbon dioxide; carboxy vinyl copolymer; carboxymethyl starch; carboxymethylamylopectin sodium; carboxymethylcellulose; carboxypolymethylene; cardamom; carmine; carmine 50; carnauba wax; carrageenan; cassia oil; castor oil; cedar leaf oil; cellacefate; cellulose; cellulose acetate; cellulose acetate α-320s and ca-398-10; cellulose microcrystalline; cellulosic polymers; cerasynt-se; and ceresin.

Also, ceteareth-12, 15, and 30; cetearyl alcohol and octanoate; ceteth-2, 10, 20 and 23; cetrimonium chloride; cetyl alcohol, esters, and palmitate; cetylpyridinium chloride; chartreus colored spheres; chemoderm 6401b; cherry; chlorobutanol; chlorobutanol hemihydrate; chlorocresol; chloroform; chloroxylenol; cholesterol; choleth; cinnamaldehyde; cinnamon; citric acid; citric acid monohydrate; clove oil; cocamide diethanolamine and ether sulfate; cocamine oxide; coco betaine; cocoa bean and butter; cocoglycerides; cocomonoethanolamide; coconut oil; coffee; collagen; compressible sugar; copovidone; coriander oil; corn glycerides; corn oil; corn oil peg-6 esters; corn syrup; cottonseed oil; cream base; creatine; creatinine; cresol; croscarmellose sodium; crospovidone; crystal gum; cupric sulfate; cutina; cyclamic acid; cyclomethicone; cysteine; and cysteine hydrochloride.

Also, dehydroacetic acid; dehymuls e; denatonium benzoate; deoxycholic acid; dextran; dextran 40; dextrates; dextrin; dextrose; diacetylated monoglycerides; diatomaceous earth; diatrizoic acid; diazolidinylurea; dibutyl phthalate; dibutyl sebacate; dichlorobenzyl alcohol; dichlorodifluoromethane; dichlorofluoromethane; dichlorotetrafluoroethane; diethanolamine; diethyl phthalate and sebacate; diethylene glycol monoethyl ether; diglycol stearate; dihydroxyaluminum glycinate; dihydroxyaluminum sodium carbonate; diisopropanolamine; diisopropyl adipate; diisopropyl dimerate; and diisopropylbenzothiazyl-2-sulfenamide.

Also, dimethicone 350, 360, copolyol, and mdx4-4210; dimethyl dioctadecylammonium bentonite, isosorbide, phthalate, siloxane, and sulfoxide; dimyristoyl lecithin; dioctylphthalate; dipropylene glycol; disodium cocoamphodiacetate, edisylate, laureth sulfosuccinate, lauryl sulfosuccinate, and sulfosalicylate; disofenin; divinylbenzene styrene copolymer; docosanol; docusate sodium; and dusting powder.

Also, edamine; edetate calcium disodium, disodium, and sodium; egg yolk phosphatides; eiderdown soap; entsulfon; epilactose; erythorbic acid; ethanolamine hydrochloride; ether; ethyl acetate, hexanediol, maltol, oleate, and vanillin; ethylcellulose; ethylene; ethylene glycol; ethylene glycol monoethyl ether; ethylene vinyl acetate copolymer; ethylenediamine dihydrochloride; ethylene-propylene copolymer; ethylparaben; eucalyptol; and eucalyptus oil.

Also, EUDRAGIT E 100, L 100, L 100-55, L 30 D, 130D-55, NE 30 D, NE 40 D, RL 100, RL 12.5, RL 30 D, RS 100, RS 12.5, RS 30 D, and S 100, and other EUDRAGIT products.

Also, eugenol; exametazime; fampridine; fatty acid esters, glycerides and pentaerythriol ester; fatty acids; fatty alcohol citrate; fatty alcohols; ferric chloride and oxide; ferrosoferric oxide; ferrous fumarate and oxide; FIRMENICH 51.226/t; florasynth; flour; fluorescein; fluorochlorohydrocarbons; formaldehyde; fructose; fumaric acid; and fused sodium ash.

Also gadolinium oxide; galactose; gamma-cyclodextrin; gelatin; gelatin 200 bloom; gellan gum; gelucire 33/01; gelva 737; gentisic acid; gentisic acid ethanolamide; ginger fluidextract; gluceptate sodium; gluceptate sodium dihydrate; gluconolactone; glucose; glucuronic acid; glutamic acid hydrochloride; glutathione; gluten; glycerin; and glycerol ester of hydrogenated rosin.

Also, glyceryl behenate, caprylate, citrate, distearate, isostearate, laurate, oleate, palmitate, palmitostearate, ricinoleate, and stearate; glycine; glycocholic acid; glycol distearate and stearate; glycyrrhizic acid; green starch blend; guanidine hydrochloride; guar gum; gum rosin; and gum.

Also HERBACOL; hetastarch; hexane; hexylene glycol; histidine; hyaluronate sodium; hydrocarbon; hydrochloric acid; hydrocortisone; hydrogel polymer; hydrogen peroxide; hydrogenated palm; hydroxyethyl cellulose and cellulose 2501; hydroxyethylpiperazine ethane sulfonic acid; hydroxymethyl cellulose; hydroxyoctacosanyl hydroxystearate; hydroxypropyl cellulose and cellulose lf; hydroxypropyl ethylcellulose 2501; hydroxypropyl methylcellulose 100, 603, 606, 2208, 2906, 2910, 4000, acetate succinate, e5, and phthalate; hydroxypropyl-b-cyclodextrin; and hystrene.

Also illicium anisatum; imidurea; insulin beef and pork; invert sugar; iodine; iodoxamic acid; iofetamine hydrochloride; irish moss extract; iron subcarbonate; isobutane; isobutyl alcohol; isoceteth-20; isoleucine; isomalt; isooctylacrylate; isopropyl alcohol, isostearate, myristate, palmitate, and stearate; isostearic acid; and isostearyl alcohol.

Also jelene; kaolin; karaya gum; kathon cg; kathon cg ii; kola nut extract; lac resin; lactate; lactic acid; lactitol monohydrate; lactobionic acid; lactoferrin; lactose; lactose monohydrate; landalgene; landalgine p; laneth; lanolin; lanolin alcohols; lanolin cholesterols; lanolin nonionic derivatives; lauralkonium chloride; lauramine oxide; laurdimonium hydrolyzed animal collagen; laureth sulfate; laureth-4 and 23; lauric diethanolamide and myristic diethanolamide; lauroyl polyoxylglycerides; lauryl sulfate; lavender; lecithin; lemon oil; leucine; levomenthol; lidofenin; light mineral oil; lime oil; limonene; lipocol sc-15; lithium hydroxide monohydrate; locust bean gum; lubritab; and lysine.

Also, magnesium acetate, aluminum silicate, aluminum silicate hydrate, aspartate, carbonate, chloride, hydroxide, nitrate, oxide, phosphate, silicate, stearate, sulfate, tartrate, and trisilicate, and other magnesium compounds.

Also, maleic acid; malic acid; maltitol; maltodextrin; maltol; maltose; mannitol; mannitol 60, 2080, and m300; mannose; maprofix; mebrofenin; medronate disodium; medronic acid; meglumine; melojel; menthol; metaphosphoric acid; methacrylic acid copolymer; methanesulfonic acid; methionine; methoxypolyoxyethylene glycol 350; methyl acrylate-methyl methacrylate; methyl alcohol, boronic acid, chloride, and ethyl ketone; methyl gluceth-10, 20, and 120 dioleate; methyl glucose sesquistearate, hydroxyethyl cellulose, laurate, paraben, salicylate, and stearate; methylated spirits; methylcellulose 400, 1500 and 4000; methylchloroisothiazolinone; methylene blue; methylene chloride;

methylisothiazolinone; methylphenidate; microcrystalline wax; mineral oil; mineral spirits; mistron spray talc; monoglycerides; monosodium citrate; montan wax; mullein leaf; multisterol extract; muscatel wine; myristic acid; myristyl alcohol and lactate; and myristyl-gamma-picolinium chloride.

Also, n,n-bis(2-hydroxyethyl)stearamide; n,n-dimethyl lauramine oxide; n,n-dimethylacetamide; n-3-chloroallyl-methenamine chloride; naphtha; n-decyl-methyl sulfoxide; neutral oil; niacinamide; nioxime; nipasept; nipastat; nitric acid; nitrogen; n-lauroylsarcosine; nonoxynol iodine; nonoxynol-9 or 15; non-pareil seeds; norflurane; n-propyl orthosilicate; and nutmeg oil.

Also oatmeal; octadecene-1/maleic acid copolymer; octanoic acid; octoxynol-1, 9, and 40; octyl hydroxystearate; octyldodecanol; octylphenol polymethylene; oil cream soda; oleic acid; oleth-2, 5, 10, and 20; oleyl alcohol and oleate; olive oil; orange juice; orange oil; orange peel extract; orvus k liquid; oxidronate sodium; and oxyquinoline.

Also palm kernel oil; palm oil; palmitamine oxide; palmitic acid; parabens; paraffin; peanut oil; pectin; PEG 6-32 stearate, sorbitan isostearate, vegetable oil, -22 methyl ether, -25 propylene glycol stearate, -40 sorbitan diisostearate, -45/dodecyl glycol copolymer, and -8 caprylic/capric glycerides; peglicol-5-oleate; pegoxol 7 stearate; pentadecalactone; pentaerythritol cocoate; pentasodium triphosphate; pentetate calcium trisodium; pentetate pentasodium; pentetic acid; peppermint; perflutren; petrolatum; petroleum distillates; PHARMABURST b1; PHARMABURST b2; pharmaceutical glaze; PHARMATOSE dcl ii; phenethyl alcohol; phenol; phenonip; phenoxyethanol; phenylalanine; phenylmercuric acetate and nitrate; phosphatidyl glycerol; phospholipid; phospholipon 90g; phosphoric acid; pine needle oil; pineapple; piperazine; piperazine hexahydrate; placebo; PLASTIBASE-50w; polacrilin; polish wax 7625 p 100; polishing solution im-182; poloxamer 124, 181, 182, 188, 237, 331, and 407; polybutene; polycarbophil; polydextrose; polydextrose k; polyester; polyester fluorocarbon diacrylate, polyamine copolymer, and rayon; and polyethylene.

Also polyethylene glycol 200, 300, 300-1600, 400, 540, 600, 800, 900, 1000, 1450, 1500, 1540, 3350, 3500, 4000, 4500, 6000, 7000k, 8000, and 20000, and other polyethylene products.

Also, polyethylene oxide; polyethylene oxide 200k, 7000k, t, and terephthalates; polygalacturonic acid; polyglactin; polyglyceryl-10 oleate and tetralinoleate; polyglyceryl-3 oleate; polyglyceryl-4 oleate; polyhydroxyethyl methacrylate; polyisobutylene; polyisobutylene 35,000 and 1,200,000; polylactide; polyols; polyoxyethylene alcohols; polyoxyethylene fatty acid esters; and polyoxyethylene propylene.

Also, polyoxyl 2, 8, 20, 40, 50, 100 and 400 stearate, 4 dilaurate, 6 isostearate, 6 and 8 laurate, 6 and 32 palmitostearate, 15 cocamine, 20 cetostearyl ether, 35, 40 and 60 castor oil, 100 glyceryl stearate, 150 distearate, and 75 lanolin; polyoxyl distearate, glyceryl stearate, lanolin, palmitate, and stearate.

Also, polyoxylethylene isononylphenyl ester; polypropylene; polypropylene glycol; polyquaternium-1, 7, and 10; polysaccharides; polysaccharides soy; polysiloxane; polysorbate 20, 40, 60, 65 and 80; polyurethane; polyvinyl acetate, acetate phthalate, alcohol, acetal, pyridine, and pyrrolidone ethylcellulose; poppy seed oil; and potash.

Also, potassium acetate, bicarbonate, bisulfite, bitartrate, carbonate, chloride, citrate, hydroxide, metabisulfite, metaphosphate, soap, and sorbate, and other potassium compounds.

Also, povidone acrylate copolymer, hydrogel, k17, k25, k26/28, k29-32, k30, k90, and k90f; povidone/eicosene copolymer; PPG-12/smdi copolymer, -15 stearyl ether, -20 methyl glucose ether distearate, and -26 oleate; primajel; proline; promalgen type D and G; propane; propellant a-46; propenyl guaethol; propyl gallate; propylene carbonate; propylene glycol alginate, diacetate, dicaprylate, monolaurate, monostearate, palmitostearate, and ricinoleate; propylparaben; prosolv 50, 90, smcc 50 and 90; protamine sulfate; protein hydrolysate; and pvm/ma copolymer.

Also, quaternium-15 and 52; quatrimycin hydrochloride; quso f-22; ra-2397; ra-3011; rhodigel-23; rosin; saccharin; saccharin calcium and sodium; safflower oil; satialgine h; SD alcohol 3a, 40, 40-2, 40b; sea spen; serine; sesame oil; shellac; shellac p.v.p. solution no. 4; silicon; silicon dioxide; silicone; silicone emulsion; simethicone; simethicone c and mdx4-4036; sipon l-20; and soap.

Also, sodium acetate, alginate, alkyl sulfate, aminobenzoate, ascorbate, benzoate, bicarbonate, bisulfate, bisulfite, bitartrate, borate, borate decahydrate, carbonate, carbonate decahydrate, carbonate hydrate, carboxymethyl betaglucan, caseinate, cellulose, cetearyl sulfate, chlorate, chloride, chlorite, cholesteryl sulfate, citrate, cocoyl sarcosinate, cyclamate, cysteinate hydrochloride, desoxycholate, dithionite, dodecyl benzene sulfonate, formaldehyde sulfoxylate, gluconate, hexametaphosphate, hydroxide, hypochlorite, iodide, lactate, laureth sulfate, lauroyl sarcosinate, lauryl sulfate, lauryl sulfoacetate, metabisulfite, nitrate, phosphate, phosphate dihydrate, polyacrylate, propionate, pyrophosphate, pyrrolidone carboxylate, starch glycolate, stearate, stearyl fumarate, succinate, sulfate, sulfate decahydrate, sulfite, tartrate, thioglycolate, thiomalate, thiosulfate, trimetaphosphate, and xylenesulfonate, and other sodium compounds.

Also, SOLULAN; SOMAY 44; sorbic acid; sorbitan monolaurate, monooleate, monopalmitate, monostearate, sesquioleate, and trioleate; sorbitol; sorbitol anhydride; soybean flour and oil; spearmint; spermaceti; spirits of turpentine; squalane; stannous chloride, fluoride, octoate, and tartrate; starch; starch 21, 825, 826, 1500 pregelatinized, 1551, and 7150; starch aluminum octenyl succinate; stearalkonium chloride; stearamidoethyl diethylamine; steareth-2, 10, 20, 21 and 100; stearic acid; stear-o-wet c; stear-o-wet m; stearoxytrimethylsilane; stearoyl polyoxylglycerides; steartrimonium hydrolyzed animal collagen; stearyl alcohol and citrate; strawberry; succimer; succinic acid; sucralose; sucrose; sucrose distearate, palmitate, polyesters, stearate, and syrup; sugar confectioners; sugar fruit fine; sulfacetamide sodium; sulfur dioxide; sulfuric acid; sulfurous acid; SURELEASE e-719010 clear; SURELEASE e-7-7050; SURFACTOL qs; SYNCHRON oral carrier; SYNCHRON oral carrier base kf; and SYNCHRON oral carrier vehicle type em.

Also, tagatose; talc; talc triturate; talcum powder; tall oil; tallow glycerides; tartaric acid; t-butyl hydroperoxide; t-butylhydroquinone; tenox; tenox-2; terpene resin; tetrachloroethylene; tetrofosmin; theophylline; thimerosal; thioglycerol; threonine; thymol; timing solution clear n-7; tin; titanium dioxide; tocopherol; tocophersolan; tolu; tragacanth; triacetin; tribehenin; tricaprylin; triceteareth-4 phosphate; trichloroethylene; trichloromonofluoromethane; trideceth-10; triethyl citrate; trifluoroacetic acid; triglycerides; trihydroxystearin; trilaneth-4 phosphate; trilaureth-4 phosphate; trimyristin; triolein; trisodium citrate dihydrate; trisodium hedta; tristearin; triton 720; trolamine; tromethamine; tryptophan; tyloxapol; and tyrosine.

Also, UNION 76 AMSCO-res 6038; urea; valine; vanillin; vegetable oil and shortening; velvetine black powder; versetamide; vinyl acetate; viscarin; viscose/cotton; vitamine; wax; wecobee fs; wheat flour; white ceresin wax; xanthan gum; xylitol; zarzarol; zein; zeolex; zinc; zinc acetate, carbonate, chloride, oxide, stearate, and sulfate, and other zinc compounds, and other agents used to dissolve, dilute, bind, fill, disintegrate, lubricate, emulsify, carry, suspend, acidify, alkalinize, buffer, solubilize, clarify, gel and/or thicken, enhance viscosity, coat, antioxidize, chelate, stabilize, modify release, and/or preserve the product.

To the extent that the foregoing lists employ trade names, it is believed that each listed trade name is well known in the industry and satisfactorily defined in the literature. The foregoing lists of excipients were generated from the U.S. Food and Drug Administration's Center for Drug Evaluation and Research's database of inactive ingredients present in FDA-approved drugs. See http://www.fda.gov/cder/iig/IIG-download.htm (accessed Nov. 27, 2007). “The Inactive Ingredients Database provides information on inactive ingredients present in FDA-approved drug products. This information can be used by industry as an aid in developing drug products.” http://www.fda.gov/cder/iig/iigfaqWEB.htm#what is active ing (accessed Dec. 4, 2007). Further information on various ingredients can be found in various resources known to those of ordinary skill in the art, including on the Internet at: http://www.usactives.com/z2/index.html (accessed Dec. 4, 2007).

### Solid Dose Excipient Modules

One possible use for solid dose excipient modules is to add volume to the API module(s) used in the final drug product. For example, one final drug product may use two API modules, and a second final drug product may use four API modules each having the same volume. To minimize costs, a standard-sized capsule may be used for both final drug products, with the space remaining after the contents of the API modules are inserted filled by one or several excipient modules. The size (volume) of such solid dose excipient modules may be determined by analysis of the volumes of the portfolio of solid dose API modules and the total volumes of the final formulation(s) (e.g., capsules) used. A variety of analytic methods can be used, ranging from trial and error number substitution to proprietary software applications, but a relatively efficient method is to simply use equation-solving subroutines available in commercial spreadsheet applications.

Once the volumes are selected, the components are developed. These solid dose ‘filler’ modules may be placebo modules composed of granules of coated non-toxic filler, developed with methods known in the art.

### Liquid Dose Excipient Modules

Multiple types of liquid dose excipient modules can be used. First are taste-masking modules used for oral administration, which contain one or more flavors, sweeteners, and other taste-masking agents. A portfolio of taste-masking modules includes several different flavors, both sweet (e.g., cherry, orange, grape, etc.) and non-sweet.

Modules with colorants can be useful to add to taste cues as well as assist final drug product differentiation. For example, a patient who is required to take one medication or mixture in the morning, and a different medication or mixture in the evening, can, in addition to different labels, have the medications customized into different colors, one for morning and the other for evening. Some final drug products optimally have no color, because it is unnecessary or creates potential problems (e.g., dye sensitivity), and separation of color modules enhances this flexibility. A limited number of colorants with maximum compatibility are selected, in some embodiments.

Modules with viscosity agents may be used to modify the thickness of the final product to affect swallowing and mouthfeel. Thicker liquids can be used for patients who have difficulty swallowing by slowing the transit of the liquid in the mouth and pharynx. On the other hand, thin liquids could be needed if the drug product is administered by feeding tube. Separation of the viscosity agent modules enhances this flexibility. A limited number of viscosity agents with maximum compatibility may be selected.

Modules with preservatives or antiseptics are useful if the final drug product is intended for multiple dose use, or for longer shelf life. These excipients, however, may complicate manufacturing due to poor solubility or interference with analytic methods, and consumers often prefer preservative-free preparations. Many modular products can be unit doses and/or intended for very short storage periods. Thus, separation of preservative modules enhances the flexibility to eliminate these from the final drug product. A limited number of preservatives with maximum compatibility can be selected.

Simply developing a module for every possible excipient would be possible, but would be expensive and might provide little advantage over current development and manufacturing techniques. To achieve cost and manufacturing benefits, the range of possible modules may be pruned to match the requirements of the portfolio of APIs and final product characteristics (e.g., color, viscosity) desired (see Module Portfolio Management below).

### Module Portfolio Management

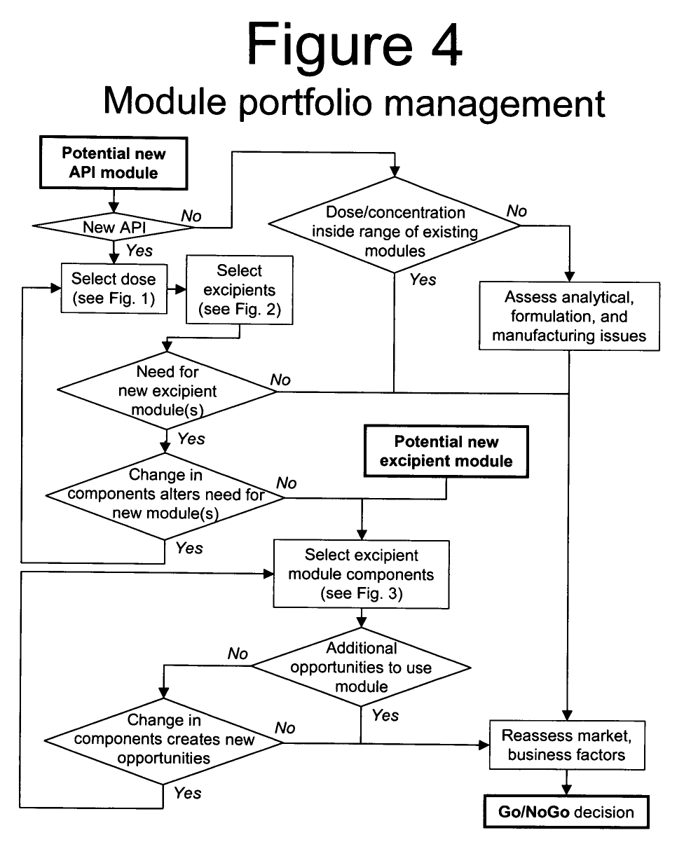

FIG. 4 summarizes the process of managing a portfolio of modules, in one embodiment of the present invention. Once portfolios of API and excipient modules have been developed, the process of adding new modules may be complex. Unlike current development and manufacturing techniques, where drug products are relatively independent except for marketing concerns, the selection of new modules may be highly dependent on the existing portfolio of modules.

When a potential new API module is identified, one possible consideration is whether the module contains an additional active ingredient. Some new API modules will simply add an additional dose or concentration of an existing active ingredient to the portfolio. In this case, if the dose/concentration is inside the range of existing modules, development is generally straightforward, using existing data and formulation knowledge. When the dose/concentration is outside the range of existing modules, additional information about analytical methods, formulation, and manufacturing may be developed or found in public records. Once these issues are defined, and full cost estimates generated, the market and business issues are reassessed and a Go or No-go decision reached, in some embodiments.

When the active ingredient in the prospective API module is new to the portfolio, additional evaluation may be required. First, the dose/concentration and excipients in the API module can be selected as outlined before. Next, the portfolio of excipient modules can be matched to the prospective API module to assess whether a new excipient module is required. If not, the cost estimates can be used in conjunction with the market analysis to reach a Go or No-go decision.

If one or more new excipient modules are needed to complement the prospective API module, the components selected for the API module may be reevaluated to assess whether changes in those components can eliminate the need for some or all of the new excipient module requirements. If so, the dose/concentration and excipients in the API module can be modified. If not, the components of the excipient module(s) may be selected as outlined before.

Once the new excipient module is designed, it can be evaluated in the context of the entire module portfolio to determine whether there are opportunities for the module to be used in conjunction with existing modules. If not, the components selected for the excipient module may be reevaluated to assess whether changes in those components can open new opportunities for synergy. Finally, the cost estimates for both the prospective API module plus any additional needed excipient modules may be used to reassess the business issues and a Go or No-go development decision can be reached.

Prospective new excipient modules can be similarly assessed against the entire current portfolio to optimize component selection and maximize opportunities for synergistic use. In a given module development endeavor, an iterative process may be used for designing and assessing potential module characteristics.

### Module Selection for Customized Final Drug Assembly

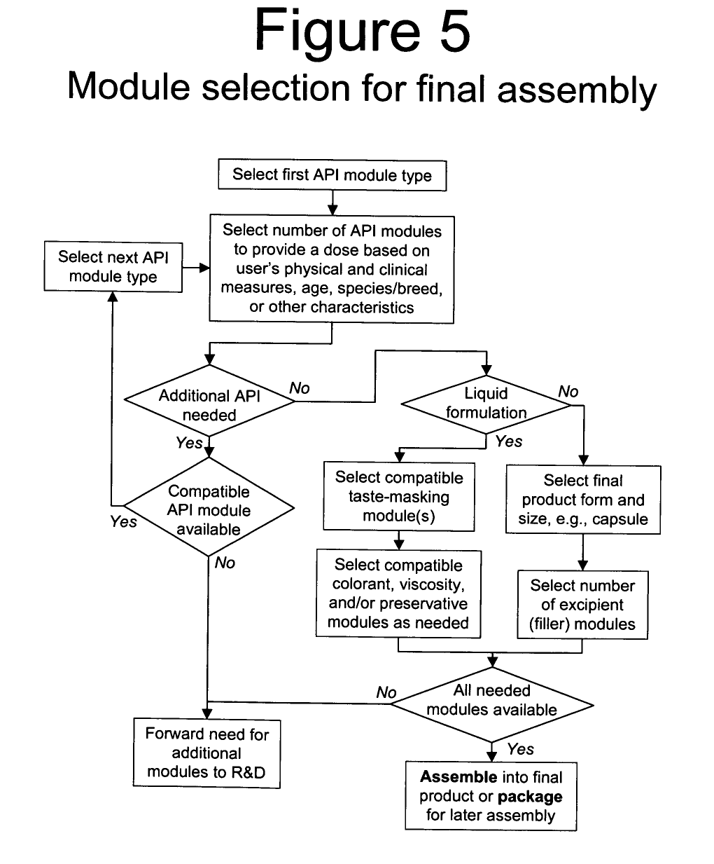

FIG. 5 summarizes the process of selecting modules for final drug product assembly, in one embodiment of the present invention. The selection of modules for assembly of a final drug, customized for the user's needs and preferences, can be straightforward. The first API module can be selected based on the needed active ingredient. The number of those modules determines the dose of the final product, which can be customized in accordance with the user's physical and clinical characteristics, age, and, in the case of animals, the species and breed. Physical characteristics that can be used to customize the dose include weight, mass, and other volumetric measures, skin surface and other area measures, and percentage body fat or lean mass and other body composition measures. Clinical characteristics that can be used to customize the dose include the user's metabolism, hepatic (liver) function, and renal (kidney) function that are measured by clinical/veterinary laboratory testing.

If additional active ingredients are needed, the module portfolio may be searched for an API module with the appropriate active ingredient that is compatible with the first API module (see below for a discussion of compatibility determination). If none is found, and an alternative API module is not available for the first active ingredient, the desired final drug combination may not be possible with the existing portfolio. For portfolio management purposes (see above), the information about the desired final drug product may be forwarded to R&D to generate a prospective API module.

If a compatible combination of API modules is available, the number of each API module may be set as described above in an iterative process until all active ingredients are selected. If the API modules are for a solid dose formulation, the final product form (e.g., capsule) and size can be selected according to methods known in the art and the appropriate number of excipient (e.g., filler) modules chosen to fill any empty space. The final drug product can be assembled immediately, or the modules packaged together as a final drug package for later assembly into a final drug product.

If the API modules are for a liquid dose formulation, compatible excipient modules can be selected. Generally, the taste-masking module may be chosen first, followed by compatible colorant, viscosity, and preservative modules as and if needed. The final drug product can be assembled immediately, or the modules packaged together for later assembly.

In both solid and liquid dose formulations, if necessary excipient modules are not available, and the API and/or other excipient modules cannot be adjusted to create a compatible combination, the information about the desired final drug product may be forwarded to R&D to generate a prospective excipient module.

### Assembly of Modules for Customized Final Drug Product

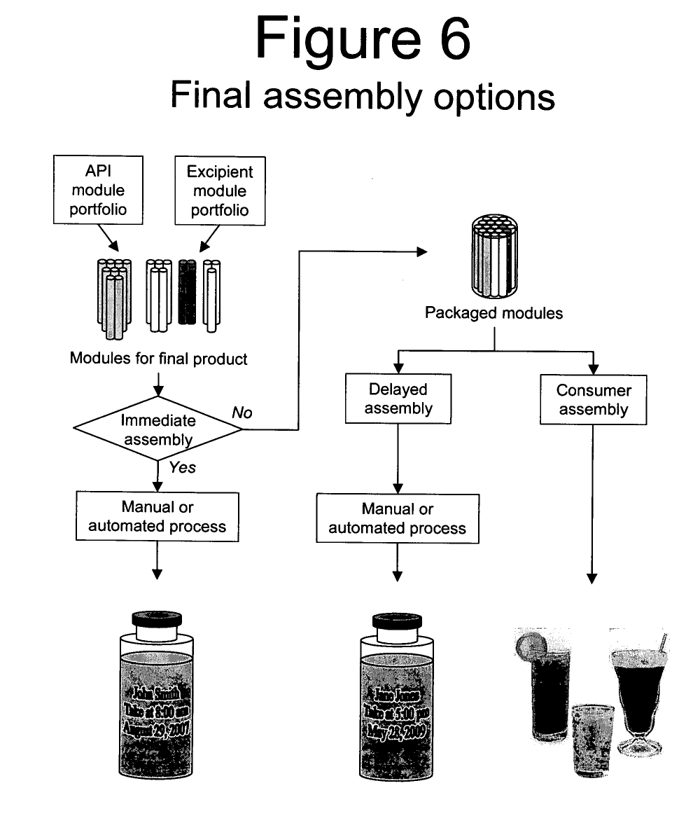

FIG. 6 summarizes the options for assembling modules for the final drug product for one embodiment. Once the API and excipient modules are selected, they can be assembled into the final drug product in many ways. The modules can be assembled and labeled immediately, by a manual or automated process, and delivered to the user. Or the modules can be packaged together for delayed assembly. Delayed assembly can be by a manual or automated process, similar to immediate assembly. Alternatively, the modules can be packaged into a kit appropriate for consumer assembly. The kit may include directions for assembly including compatible home ingredients. Depending on the components and compatibility of the modules, assembly can include mixing the modules with non-pharmaceutical liquids such as fruit juices, soft drinks, hot beverages, or other liquids for consumption.

### Quality Control

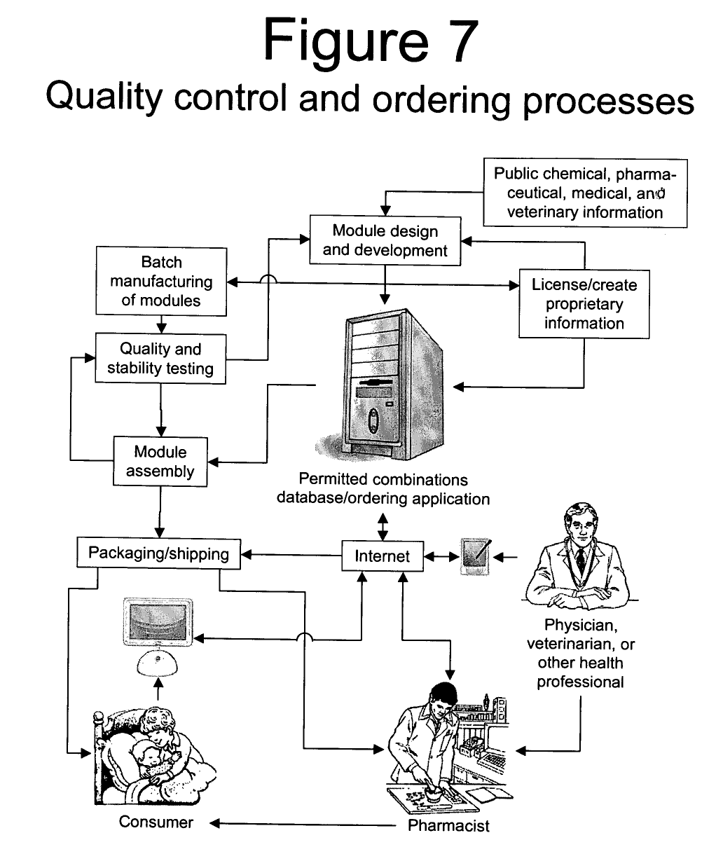

FIG. 7 summarizes the methods of controlling quality, including assessing compatibility and permissible combinations of modules, and ordering processes, in one embodiment of the present invention.

Specific modules, whether API or excipient, can be manufactured in a batch process and quality may be tested using analytic methods and stability protocols known to the art. The data from quality testing, as well as public and proprietary chemical, pharmaceutical, medical and veterinary information about the various components of the modules and their interactions, may be used to guide and refine module design and development. And that knowledge may be further used to refine module manufacturing and create a computer database of permitted combinations of modules.

The database of permitted combinations of modules can be maintained on a computer and may be linked to both an ordering application and the Internet or other computer network. Integration of the database and ordering application enhances quality by controlling the assembly of the final drug products and preventing the combination of incompatible modules, in some embodiments. Linking to the Internet allows health professionals or consumers to customize and order the final drug product, in other embodiments.

The ordering and fulfillment process can occur in multiple ways. Physicians, veterinarians, and other health professionals with prescribing privileges can access the database by the Internet and select the active ingredient(s), dose(s), form, and other characteristics of the final drug product, in some embodiments. The prescription can be forwarded electronically to either a pharmacist or the patient/consumer, who can order and purchase the drug using methods known in the art. Once ordered, the drug product can be packaged and shipped to the patient/consumer, either directly or via the pharmacist.

Alternatively, the consumer can access the database by the Internet and select the active ingredient(s), dose(s), form, and other characteristics of the final drug product, in other embodiments. Non-prescription and nutritional products can be ordered and purchased by the consumer, and shipped directly to him. The consumer can also initiate the fulfillment process by forwarding the desired drug and/or characteristics to a physician, veterinarian, or other licensed professional for approval and prescription.

Finally, the customized products can be labeled with the name, date, and specific time of administration of each dose. Standard multidose containers of capsules or solutions are labeled with the dose and frequency of administration (e.g., twice a day). Patients, however, often forget whether they have taken the drug, and the standard container gives few clues to assist their memory. Various methods, such as weekly pillboxes with multiple compartments, have been developed to help drug compliance, but all these have deficiencies. Modular precursors, though, allow multiple ingredients and doses to be combined and taken at once in some embodiments of the present invention. The consumer's drug can thus be organized by the specific time of administration, rather than by active ingredient, which enhances compliance.

Although not required, the invention will be described in the general context of computer-executable instructions, such as program modules, being executed by a personal computer. Generally, program modules include routines, programs, objects, components, data structures, etc., that perform particular tasks or implement particular abstract data types. Moreover, those skilled in the art will appreciate that the invention may be practiced with other computer system configurations, including hand-held devices, multiprocessor systems, microprocessor-based or programmable consumer electronics, network PCs, minicomputers, mainframe computers, and the like. The invention may also be practiced in distributed computing environments where tasks are performed by remote processing devices that are linked through a communications network. In a distributed computing environment, program modules may be located in both local and remote memory storage devices. U.S. Pat. No. 7,243,299, which is incorporated herein by reference, includes a detailed description of a conventional general-purpose digital computing environment that can be used to implement various aspects of the invention.

In some embodiments of the present invention, methods of selecting one or more API modules and one or more excipient modules, and manufacturing final drug packages and final drug products are performed by computer-executable instructions stored on one or more computer-readable media. Computer-readable media are any suitable media that can store data, including instructions, that are accessible by a computer. Computer-readable media, such as hard disks, removable magnetic disks, removable optical disks, magnetic cassettes, flash memory cards, digital versatile disks, Bernoulli cartridges, random access memories (RAMs), read only memories (ROM), and the like, may be used in some embodiments of the present invention.

A “computer” includes any suitable machine. Such machines include, but are not limited to, desk top computers, lap top computers, palm top computers, PDAs, cellular telephones, media players (such as, for example, iPODS and KINDLES), servers, dedicated apparatus such as equipment adapted to manufacture one or more of modules, final drug packages, and final drug products, and the like. In some embodiments, a computer includes more than one machine working in concert with another. Examples of multiple machines working together include, but are not limited to, a PDA accessing a database stored on a server through a broadband connection via the Internet; a terminal accessing a mainframe computer; and a desk top computer accessing a network of other computers.

Computer-executable instructions and computer-executable components appearing in some embodiments of the present invention include software programs, operating systems, applications, subroutines, data structures, and the like. Such instructions and components may be stored on a single memory device, or on many memory devices, and may be local (e.g., on a single computer), distributed (e.g., on more than one computer), remote (e.g., accessed through a network or the Internet), or a combination thereof. Similarly, those instructions and components can be operated locally, in a distributed manner, remotely, or a combination thereof. For example, one computer in a doctor's office could correlate patient information including one or more of diagnosis, prescription, age, weight, gender, allergy information, and aesthetic preferences with one or more remote computer systems that contain API module information and excipient module information to develop a final drug product.

A system for manufacturing at least one final drug product, at least one final drug package, or both, in some embodiments, comprises at least one API module portfolio and at least one excipient module portfolio, in which the portfolios are operatively connected to manufacture the final drug product or final drug package. In some embodiments, “operatively connected to manufacture the final drug product or final drug package” means the system is capable of following instructions for selecting a number of API modules from the API module portfolio, and selecting a number of excipient modules from the excipient module portfolio to form the final product or package. In some embodiments, the system can form a single dose or multiple doses from the selected modules. The system, in other embodiments, comprises one or more module storage facilities, optionally one or more module packaging structures for making a final drug package, and optionally one or more module content combination means for making a final drug product. The system can be a manual system, an automated system, or a partially automated system. In other embodiments, the system is capable of determining the number of various modules necessary to formulate a final drug product, or to assemble a final drug package, based on inputted prescription information.

Other embodiments of the present invention provide systems and methods for manufacturing one or more API modules, one or more excipient modules, or a combination thereof. Such systems, in some embodiments, are adaptable to combine a fixed amount of at least one API with another fixed amount of at least one excipient, to make at least one API module. Other embodiments provide systems to isolate a fixed amount of at least one excipient to make at least one excipient module. In still other embodiments, such systems are adaptable to combine a fixed amount of one excipient with another fixed amount of another excipient to make at least one excipient module. Yet other embodiments are adaptable to deliver the contents of the module to a container. The system can be a manual system, an automated system, or a partially automated system. In some embodiments, the system is capable of determining one or more fixed amounts based on instructions received.

Some embodiments of the present invention comprise means for storing compatibility information for each API module and each excipient module. In some of those embodiments, a given module has associated compatibility information, such as which APIs, excipients, API modules, and/or excipient modules are compatible with the given module. In other embodiments, compatibility information contains concentration information, such as the relative amount of an excipient needed to solubilize a given amount of an API. Such means include any suitable means for storing information. In some embodiments, means for storing compatibility information are chosen from paper records, computer-readable records, and combinations thereof. Computer-readable records can be stored on computer-readable media. In still other embodiments, various modules are classified for easy formulation. For example, all modules in a given class can be considered compatible, and therefore may be readily combined to form a final drug product. In other embodiments, several classes are used.

It is contemplated, for some embodiments of the present invention, that proprietary information will be developed regarding, for example, the composition, amounts, and compatibility of API modules and excipient modules. In other embodiments, at least some data will be generated in an open source code fashion, whereby physicians, pharmacists, manufacturers, and/or patients develop and share expertise about compatibility among APIs, excipients, API modules, and/or excipient modules. For example, new compatibility information can be recorded, stored, updated, and consulted in a widely-accessible forum, such as one or more websites on the Internet to assist others to formulate new embodiments of the invention. In some embodiments, such a forum is peer-reviewed and managed by knowledgeable professionals who ensure the integrity of the information. In other embodiments, the forum allows patients to provide their anecdotal evidence and experience about the compatibility of various final drug packages and final drug products they have used. Professionals and patients alike can consult such forums to assist the development of new modules, new portfolios, new final drug packages, and new final drug products. Some embodiments of the present invention provide computer-executable instructions for consulting such forums during the development of new portfolios, new modules, new final drug packages, and new final drug products.

### EXAMPLES

### Example 1

### Customizing Formulation Type for a Pediatric Patient

A 5 year old male is seen by is pediatrician and diagnosed with spasticity from cerebral palsy. The spasticity is not controlled by the standard, first-line drug, which is available in an oral liquid formulation. The physician plans to add a second antispastic drug, but this drug is only available in a tablet formulation and the patient is not able to swallow tablets. The physician then prescribes a customized final drug product that is an oral liquid. First, he accesses the API module database via the Web and confirms that the desired active pharmaceutical ingredient is available. After selecting the appropriate API module and dose, he follows software-provided prompts to select the excipient modules necessary (sweetener and flavor-enhancer, colorant, preservative, viscosity agent, and additional carrier) to complete the formulation. He knows the child hates cherry, so he selects a grape flavor instead. A customized prescription is generated detailing the number of each specific module, and is signed and filed electronically with an appropriate facility for final assembly and delivery to the patient.

### Example 2

### Customizing Dose in an Elderly Patient

A 96 year-old female is seen by her gerontologist and diagnosed with depression. This has occurred before, and the physician plans to prescribe an antidepressant. In the past, the patient has been very sensitive to the sedative effects of the preferred antidepressant, but she solved the problem by splitting the lowest available dose tablets. Now, however, the patient's eyesight and dexterity have deteriorated to the point where splitting tablets is unsafe. In addition, the physician believes it is likely the patient's sensitivity has increased due to continuing decline in her liver function. Following the same procedure as in Example 1, the physician prescribes a customized drug that includes the desired active ingredient in a capsule with one quarter of the lowest commercially available dose. The customized prescription is generated and sent to a local pharmacy for final assembly, and the patient is able to pick up the medication later that day. Two weeks later, the patient reports less than optimal improvement, and since she is not experiencing any sedative side-effects, the physician changes the customized drug to one containing 35% of the lowest dose available commercially (a dose that would be impossible to duplicate with tablet splitting).

### Example 3

### Customizing Excipients to Minimize Allergic Reaction

A 47 year-old female is seen by her gynecologist and diagnosed with vaginal bacteriosis. The physician plans to prescribe a topical cream, but the patient is allergic to the commercially available formulation, specifically, the viscosity agent used to enhance the ‘creaminess’ of the product. The physician then prescribes a customized drug that includes the desired active ingredient, but without the allergen. The physician accesses the module database and selects the appropriate modules following the same procedure as in previous examples. In this case, a formulation is possible without any viscosity agent at all. The physician's office maintains a drug assembly kiosk for patient convenience, and since all needed API modules and excipient modules are on hand, the patient is able to leave the office with her medication.

### Example 4

### Simplifying a Medication Regimen in an Elderly Patient

A 75 year-old male is seen by his internist in a follow-up visit. He has numerous medical problems, including hypertension, hypercholesterolemia, heart disease, and diabetes, and is on eight different medications. The patient's blood pressure is high, and he complains to his physician that he is having a hard time keeping track of all the medications. Upon checking, the physician discovers that the patient neglected to fill one prescription for hypertension. The physician accesses the module database and learns that 6 of the 8 prescribed drugs are available in API modules. Following an interactive interface he selects the six APIs, indicates the desired dosage, and is presented with the option of combining three of the medications into one daily capsule, and combining the other three drugs into another capsule taken twice a day. The two customized prescriptions are generated, which simplify the patient's drug regimen from a total of 15 tablets taken at three different times of the day, to only four tablets at breakfast and two more at dinner. The two remaining prescribed drugs, for which API modules are not yet available, are taken in conventional form.

### Example 5

### Preventing Drug Incompatibilities in a Hospitalized Patient

A 58 year-old female is admitted to Hospital A with advanced breast cancer for a course of chemotherapy with three agents. At the same time another patient is admitted to Hospital B for the same, well accepted but relatively rare treatment plan. As standard practice, all three chemotherapy agents are sequentially attached to the same intravenous line. Unbeknownst to the hospitals or medical professionals, however, the manufacturer of one chemotherapy agent has changed its formulation slightly to use a different excipient. The new formulation is well tested for compatibility with frequently used treatment protocols and approved by the FDA because it stabilizes the active ingredient and permits a longer shelf life. It has not, however, been tested with the treatment protocol planned for these two patients.

In Hospital A, where each drug is purchased separately, when the chemotherapy is administered, the new formulation interacts with the other, unchanged agents, and precipitates, causing severe complications. In Hospital B all multiagent treatments are customized using precursor modules and assembled in the hospital's pharmacy. When the desired agents are selected, the potential incompatibility is highlighted as an untested combination. The pharmacist is given a choice of including the older version of the API module in a triple combination, or to generate two customized drugs with instructions that prevent concurrent administration of the new version of the API module. The older version in triple combination is selected, and the patient has an excellent response.

### INDUSTRIAL APPLICABILITY

Some embodiments of the present invention relate to the manufacture of medicaments useful for treating one or more diseases, disorders, and conditions in one or more human and animal patients in need thereof. In some aspects, the present invention improves on conventional methods of manufacturing drugs, which depend on economies of scale. Those economies of scale require relatively large patient populations, because the regulatory burdens of bringing a new drug to market render so-called “orphan drugs” economically infeasible. Accordingly, some embodiments of the present invention provide economically viable means to manufacture final drug products for underserved patient populations.

In other aspects, the present invention industrializes so-called formulary pharmacy, in which a pharmacist formulates a prescribed medicine for an individual patient. Formulation accuracy, potency preservation, contaminant and microbial control, and shelf life are greatly enhanced over that achievable by formulary pharmacy in some embodiments of the present invention. Moreover, individual patients and health care providers are given much greater control over the medicines, vitamins, botanicals, nutraceuticals, and other substances to be consumed for health purposes.

While the invention has been particularly shown and described with reference to preferred embodiments thereof, it will be understood by those skilled in the art that various changes in form and details may be made therein without departing from the spirit and scope of the invention. Furthermore, the foregoing description of various embodiments does not necessarily imply exclusion. For example, “some” embodiments may include all or part of “other” and “further” embodiments within the scope of this invention.

What is claimed is:

1. A method for manufacturing a final drug product, comprising:
selecting a first API module comprising
at least one first active pharmaceutical ingredient and
at least one first excipient;
determining a number of the first API module;
choosing a first excipient module comprising
at least one second excipient;
determining a number of the first excipient module; and
combining the number of the first API module and the number of the first excipient module to manufacture the final drug product.

2. The method of claim 1, wherein:
the number of the first API module is greater than one.

3. The method of claim 2, wherein:
the number of the first API module is greater than five.

4. The method of claim 1, implemented by computer-executable instructions stored on one or more computer-readable media.

5. The method of claim 1, further comprising:
selecting a second API module comprising
at least one second active pharmaceutical ingredient and
at least one third excipient;
determining a number of the second API module;
combining the number of the second API module with the number of the first API module and the number of the first excipient module to manufacture the final drug product.

6. The method of claim 5, further comprising:
selecting a second excipient module comprising
at least one fourth excipient;
determining a number of the second excipient module; and
combining the number of the second excipient module with the number of the second API module, the number of the first API module, and the number of the first excipient module to manufacture the final drug product.

7. The method of claim 5, implemented by computer-executable instructions stored on one or more computer-readable media.

8. A final drug product, made by a process comprising:
combining at least one API module comprising at least one active pharmaceutical ingredient, and at least one excipient module, to make the final drug product;
wherein the at least one active pharmaceutical ingredient is present in the final drug product in an amount effective for treating at least one disease, at least one disorder, at least one condition, or a combination thereof, in at least one human or animal patient in need thereof.

9. A final drug package, comprising:
at least one API module, which comprises
at least one first excipient, and
at least one active pharmaceutical ingredient; and
at least one excipient module, which comprises
at least one second excipient;
wherein the at least one second excipient is compatible with the at least one active pharmaceutical ingredient;
wherein the at least one active pharmaceutical ingredient is present in the final drug package in an amount effective for treating at least one disease, at least one disorder, at least one condition, or a combination thereof, in at least one human or animal patient in need thereof; and
wherein the at least one API module and the at least one excipient module are adapted to form a final drug product.

10. An API module, comprising:
at least one excipient in a first fixed amount, and
at least one active pharmaceutical ingredient in a second fixed amount of at least a dose escalation interval or a fraction thereof.

11. The API module of claim 10, wherein the second fixed amount is greater than the dose escalation interval.

12. The API module of claim 10, wherein the second fixed amount is less than a dose of the at least one active pharmaceutical ingredient.

13. The API module of claim 12, wherein the second fixed amount is less than the minimum dose of the at least one active pharmaceutical ingredient.

14. The API module of claim 13, wherein the second fixed amount is less than about 80% of the minimum dose of the at least one active pharmaceutical ingredient.

15. An excipient module, comprising:
at least one first excipient in a first fixed amount; and
at least one second excipient in a second fixed amount;
wherein the at least one first excipient and the at least one second excipient are compatible with at least one active pharmaceutical ingredient.

16. The excipient module of claim 15, wherein the at least one first excipient and the at least one second excipient are compatible with two or more active pharmaceutical ingredients.

17. A system for manufacturing at least one final drug product, at least one final drug package, or both, comprising:
at least one API module portfolio comprising a plurality of different API modules; and
at least one excipient module portfolio comprising a plurality of different excipient modules;
wherein the at least one API module portfolio and the at least one excipient module portfolio are operatively connected to manufacture the at least one final drug product, the at least one final drug package, or both.

18. The system of claim 17, further comprising one or more means for storing compatibility information for at least one API module and at least one excipient module.

19. A computer-readable medium having computer executable components implementable in a system operable to manufacture at least one final drug product, at least one final drug package, or both, comprising:
a computer-executable component for providing one or more of compatibility information, dose information, and dose escalation interval information for at least one API module; and
a computer-executable component for providing compatibility information for at least one excipient module.
